(19) United States
(12) Patent Application Publication
(10) Pub. No.: US 2026/0015365 A1
(43) Pub. Date: Jan. 15, 2026

(54) PRODRUGS OF SOMATOSTATIN RECEPTORS SUBTYPE 4 (SSTR4) AGONISTS AND THEIR APPLICATIONS

(71) Applicant: Humanwell Pharmaceutical US, Inc., Ballwin, MO (US)

(72) Inventors: Shuo Zhao, Ballwin, MO (US); Suresh Kuarm Bowroju, Ballwin, MO (US); Subo Liao, Ballwin, MO (US); Jun Yang, Ballwin, MO (US); Hao Zhou, Yichang (CN); Zejian Ding, Yichang (CN); Xin Zheng, Yichang (CN); Rong Liu, Yichang (CN)

(21) Appl. No.: 19/265,559

(22) Filed: Jul. 10, 2025

Related U.S. Application Data
(60) Provisional application No. 63/669,910, filed on Jul. 11, 2024.

(51) Int. Cl. C07D 519/00; A61K 31/437; A61K 31/439; A61K 31/4439; C07D 401/12; C07D 471/04

(52) U.S. Cl. CPC C07D 519/00; A61K 31/437; A61K 31/439; A61K 31/4439; C07D 401/12; C07D 471/04

(57) ABSTRACT

The invention discloses a class of prodrugs of somatostatin receptor subtype 4 (SSTR4) small molecule agonists, as well as their pharmaceutical compositions, preparation methods, and uses. The SSTR4 small molecule agonist prodrug is represented by formula (I), with specific substituents and definitions as described in the specification. The SSTR4 small molecule agonist prodrug can improve pharmacokinetic properties, reduce side effects, and such compounds or their pharmaceutical compositions can be used in the treatment and/or prevention of SSTR4 receptor-related pain conditions.

### CROSS-REFERENCE TO RELATED APPLICATION

This application claims priority to U.S. Provisional Patent Application Ser. No. 63/669,910, filed Jul. 11, 2024, entitled “PRODRUGS OF SOMATOSTATIN RECEPTOR SUBTYPE 4 (SSTR4) AGONISTS AND THEIR APPLICATIONS”, the disclosure of which is hereby incorporated by reference in its entirety.

### FIELD OF THE INVENTION

The present invention specifically relates to a class of prodrugs of somatostatin receptor subtype 4 (SSTR4) small molecule agonists, as well as their pharmaceutical compositions, preparation methods, and uses.

### BACKGROUND OF THE INVENTION

Analgesics are essential medications for many patients to relieve pain and improve their quality of life. With the global aging population and the rising incidence of chronic diseases such as diabetes, arthritis, joint or bone pain, epilepsy, depression, nerve damage, and various cancers, the demand for pain treatment medications has significantly increased. Currently, the global pain treatment market is mainly dominated by opioids and non-steroidal anti-inflammatory drugs (NSAIDs). However, opioids can lead to side effects such as respiratory depression, dependency, and constipation, and the abuse of opioids can also cause societal crises. Therefore, there is a need to develop non-addictive analgesics to meet the urgent needs of patients.

Somatostatin, also known as growth hormone-inhibiting factor, is a cyclic peptide produced by various organs and tissues in the human body. It can act both systemically and locally to inhibit the secretion of various hormones, growth factors, and neurotransmitters, including insulin and glucagon. Somatostatin plays an important role in regulating cell proliferation, glucose homeostasis, inflammation, and pain. Its biological properties are mediated by the G-protein coupled receptor family of somatostatin receptors (SSTRs), which include five subtypes: SSTR1, SSTR2, SSTR3, SSTR4, and SSTR5.

The SSTR4 receptor is distributed in the axons and cell bodies of dorsal root ganglion neurons in rats, monkeys, and humans. It is generally believed that modulating the SSTR4 pathway can alleviate or inhibit pain and inflammation processes. According to recent studies, somatostatin can relieve pain even when opioids fail, playing a crucial role in neuroregulation, such as SSTR4-mediated pain control (Chrubasik J, Chrubasik S, Martin E, Acta Neurobiol Exp (Wars). 1993; 53 (1): 289-96. Penn R D; Paice J A, Kroin J S, Pain. 1992 April; 49 (1): 13-19).

Other studies have shown that in the absence of SSTR4, mice are prone to persistent pain and lack analgesic effects (Van Op den Bosch J, et al, J Cell Mol Med, 2009, 13:3283-3295; Helyes Z. et al, Proc Natl Acad Sci USA. 2009, 106:13088-13093). Therefore, selective SSTR4 agonists may provide useful therapeutic options for pain and/or inflammation (Ahmed F. Abdel-Magid, ACS med Chem Lett. 2015, 6, 110). While most SSTR subtypes are involved in regulating homeostatic hormones, SSTR4 appears to play a functional role in regulating sensory neurotransmitters (Pruyank A. Shenoy, Frontiers in Pharm. 2018, vol 9, article 495).

SSTR4 regulates dorsal root ganglion neurons in multiple ways, such as enhancing potassium currents by opening G-protein inwardly rectifying potassium channels, inhibiting calcium currents by suppressing voltage-gated calcium channels, and reducing calcium currents through transient receptor potential vanilloid-1 and ankyrin 1 channels, thereby controlling nociception.

SSTR4 small molecule agonists have been reported (Michael Ankersen, Michael Crider, Shengquan Liu, Bin Ho, Henrik S. Andersen, and Carsten Stidsen, J. Am. Chem. Soc. 1998, 120, 1368-1373; Mia Engström, Jussi Tomperi, Kamel El-Darwish, Mikaela Åhman, Juha-Matti Savola, and Siegfried Wurster, JPET, 2005, 312:332-338; A. Michael Crider and Ken A. Witt, Mini-Reviews in Medicinal Chemistry, 2007, 7, 213-220), with some showing excellent anti-inflammatory and analgesic effects (Boglárka Kántás et al; Int. J. Mol. Sci. 2019, 20, 6245; Eva Szökea, Neuropharmacology 178 (2020) 108198). Pharmaceutical companies have filed multiple patents to protect their novel SSTR4 agonists and their potential as new analgesics (U.S. Pat. No. 971,282, U.S. Pat. Nos. 9,957,267, 10,166,214, 10,577,336, US20120190691A1, US20180092880A1, WO2021233427A1, WO2021233428A1). Notably, Eli Lilly has developed compounds that have entered Phase II clinical trials. Recently, the binding structure of the SSTR4 receptor with its ligand has been reported, which will aid in better ligand design (Wenli Zhao et al. Cell Research, 2022, 0:1-12).

Patent WO2024102754A2 discloses a class of novel SSTR4 agonists that can provide effective pain treatment to meet patient needs. Here, we disclose prodrugs of these SSTR4 agonists, aiming to improve pharmacokinetics and reduce side effects.

### SUMMARY OF THE INVENTION

The following is an overview of the subject matter detailed in this invention's description. This summary is not intended to limit the scope of the claims.

The present disclosure is directed to a compound as shown in formula (I), or its stereoisomers, pharmaceutically acceptable salts, solvates, deuterated derivatives, metabolites, or prodrugs:

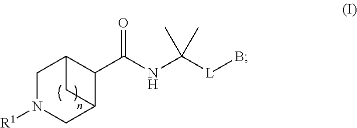

wherein:
   n=0 or 1;
   L is selected from the group consisting of a bond, —(CH₂)ₘ—, and —(CH₂)ₘO—;
   m=0, 1, 2, or 3;
   B is selected from the group consisting of an aryl ring, a substituted aryl ring, a heterocyclic ring, a substituted heterocyclic ring, an alkyl-substituted heterocyclic ring, and a substituted alkyl-substituted heterocyclic ring; the B ring is further optionally substituted with 0-6 substituents independently selected from the group consisting of hydrogen, halogen, cyano, C1-C6 alkyl, halo-C1-C6 alkyl, C3-C6 cycloalkyl, C1-C3 alkoxy, alkylsilyl, aryl, substituted aryl, and thioalkyl;
   R¹ is selected from the following:

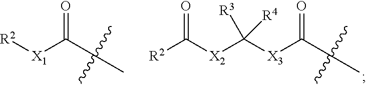

X₁ is selected from a single bond, CH₂, or O;
   X₂ and X₃ are each independently selected from CH₂ or O;
   R² is selected from C1-C10 alkyl, substituted C1-C10 alkyl, C3-C8 cycloalkyl, substituted C3-C8 cycloalkyl, aryl, substituted aryl, heteroaryl, substituted heteroaryl, heterocyclic, substituted heterocyclic; substituted C1-C10 alkyl, substituted C3-C8 cycloalkyl, substituted aryl, heteroaryl, or heterocyclic groups are substituted with 1-3 substituents independently selected from the group consisting of halogen, cyano, C1-C6 alkyl, halo-C1-C6 alkyl, C3-C6 cycloalkyl, and C1-C3 alkoxy; and

R³ and R⁴ are each independently selected from hydrogen, deuterium, and C1-C6 alkyl.

The present disclosure is also directed to a pharmaceutical composition comprising the compound of formula (I), or its stereoisomers, pharmaceutically acceptable salts, solvates, deuterated derivatives, metabolites, or prodrugs, and a pharmaceutically acceptable carrier.

The present disclosure is further directed to use of the compound of formula (I) in the preparation of a medicament for the treatment and/or prevention of diseases or disorders associated with the SSTR4 receptor.

The present disclosure is further directed to use of the compound of formula (I) in the preparation of a medicament for the treatment and/or prevention of pain conditions associated with the SSTR4 receptor.

### DETAILED DESCRIPTION

The present disclosure provides a class of prodrugs of SSTR4 agonists, their preparation methods, and their uses. The compounds of the invention offer effective pain treatment, exhibit good pharmacokinetic properties, have minimal side effects, and meet patient needs.

In one aspect, embodiments of the invention provide compounds as shown in formula (I), or their stereoisomers, pharmaceutically acceptable salts, solvates, deuterated derivatives, metabolites, or prodrugs:

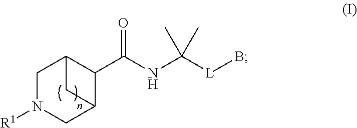

wherein, n=0 or 1;
L is selected from the group consisting of a bond, —(CH₂)ₘ—, and—(CH₂)ₘO—, m=0, 1, 2, or 3;
B is selected from the group consisting of an aryl ring, substituted aryl ring, heterocyclic ring, substituted heterocyclic ring, alkyl-substituted heterocyclic ring, and substituted alkyl-substituted heterocyclic ring; the B ring is further optionally substituted with 0-6 substituents independently selected from the group consisting of hydrogen, halogen, cyano, C1-C6 alkyl, halo-C1-C6 alkyl, C3-C6 cycloalkyl, C1-C3 alkoxy, alkylsilyl, aryl, substituted aryl, and thioalkyl; R¹ is selected from the following:

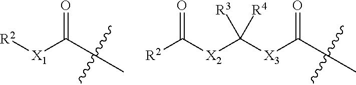

wherein,
X₁ is selected from a single bond, CH₂, or O;
X₂ and X₃ are each independently selected from CH₂ or O;
R² is selected from C1-C10 alkyl, substituted C1-C10 alkyl, C3-C8 cycloalkyl, substituted C3-C8 cycloalkyl, aryl, substituted aryl, heteroaryl, substituted heteroaryl, heterocyclic, or substituted heterocyclic; substituted C1-C10 alkyl, substituted C3-C8 cycloalkyl, substituted aryl, heteroaryl, or heterocyclic groups are further substituted with 1-3 substituents independently selected from the group consisting of halogen, cyano, C1-C6 alkyl, halo-C1-C6 alkyl, C3-C6 cycloalkyl, and C1-C3 alkoxy; and
R³ and R⁴ are each independently selected from hydrogen, deuterium, and C1-C6 alkyl.

Specifically, in some embodiments, the present invention provides a compound as shown in formula (II) or formula (III), and its stereoisomers, pharmaceutically acceptable salts, solvates, deuterated derivatives, metabolites, or prodrugs:

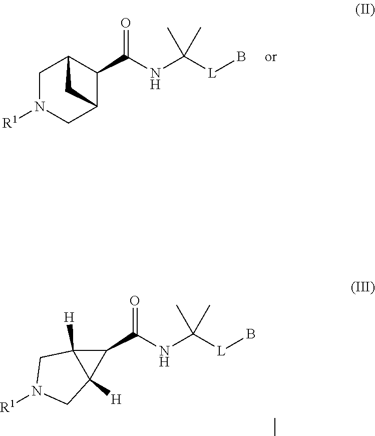

wherein
L is selected from the group consisting of —(CH₂)ₘ-and-(CH₂)ₘO—, m=0 or 1;
B is selected from the group consisting of an aryl ring, substituted aryl ring, heterocyclic ring, substituted heterocyclic ring, alkyl-substituted heterocyclic ring, and substituted alkyl-substituted heterocyclic ring; the B ring is further optionally substituted with 0-6 substituents independently selected from the group consisting of hydrogen, halogen, cyano, C1-C6 alkyl, halo-C1-C6 alkyl, C3-C6 cycloalkyl, C1-C3 alkoxy, alkylsilyl, aryl, substituted aryl, and thioalkyl; Preferably, B is selected from phenyl, naphthyl, pyridyl, pyrimidyl, and imidazopyridyl;
R¹ is selected from the following:

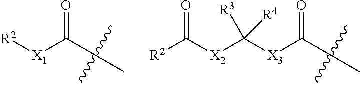

wherein,
X₁ is selected from a single bond, CH₂, or O;
X₂ and X₃ are each independently selected from CH₂ or O;
R² is selected from C1-C10 alkyl, C3-C8 cycloalkyl, substituted C1-C10 alkyl, or substituted C3-C8 cycloalkyl; substituted C1-C10 alkyl or substituted C3-C8 cycloalkyl is further substituted with 1-3 substituents independently selected from the group consisting of halogen, cyano, C1-C6 alkyl, halo-C1-C6 alkyl, C3-C6 cycloalkyl, and C1-C3 alkoxy;
R³ and R⁴ are each independently selected from hydrogen, deuterium, and C1-C3 alkyl.

Furthermore, specifically, in some embodiments, the present invention provides a compound as shown in formula (II-1), and its stereoisomers, pharmaceutically acceptable salts, solvates, deuterated derivatives, metabolites, or prodrugs:

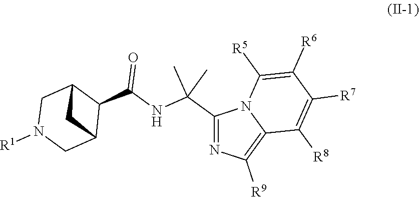

R¹ is selected from the following:

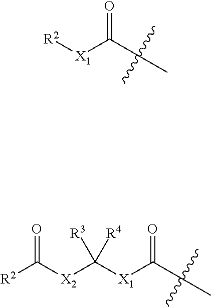

wherein,
X₁ is selected from a single bond, CH₂, or O;
X₂ and X₃ are each independently selected from CH₂ or O;
R² is selected from C1-C6 alkyl, C3-C6 cycloalkyl, substituted C1-C6 alkyl, or substituted C3-C6 cycloalkyl; substituted C1-C6 alkyl or substituted C3-C6 cycloalkyl is further substituted with 1-3 substituents independently selected from the group consisting of halogen, cyano, C1-C6 alkyl, halo-C1-C6 alkyl, C3-C6 cycloalkyl, and C1-C3 alkoxy;
R³ and R⁴ are each independently selected from hydrogen, deuterium, and C1-C3 alkyl;
R⁵, R⁶, R⁷, R⁸, and R⁹ are each independently selected from hydrogen, halogen, cyano, C1-C6 alkyl, halo-C1-C6 alkyl, C3-C6 cycloalkyl, C1-C3 alkoxy, alkylsilyl, aryl, substituted aryl, and thioalkyl.

Further preferably, in some specific embodiments, the compounds as shown in formula (II-1a) and formula (II-1b), or their stereoisomers, pharmaceutically acceptable salts, solvates, deuterated derivatives, metabolites, or prodrugs:

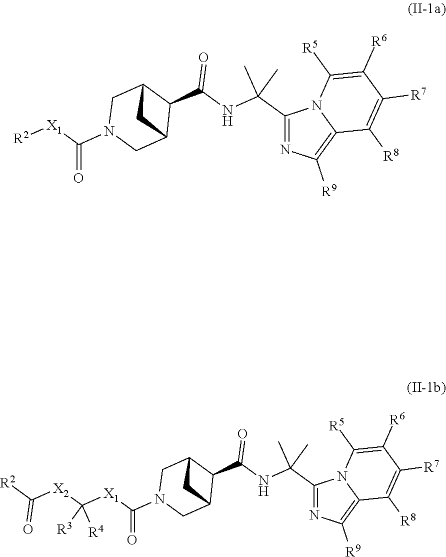

R¹ is selected from the following:

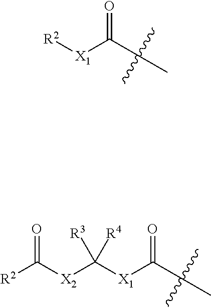

wherein,
X₁ is selected from a single bond, CH₂, or O;
X₂ and X₃ are each independently selected from CH₂ or O;
R² is selected from methyl, ethyl, propyl, isopropyl, butyl, tert-butyl, cyclopropyl, cyclobutyl, cyclopentyl, or cyclohexyl; substituted C1-C6 alkyl or substituted C3-C6 cycloalkyl is further substituted with 1-3 substituents independently selected from the group consisting of fluorine, chlorine, bromine, iodine, cyano, methyl, ethyl, propyl, fluoromethyl, difluoromethyl, trifluoromethyl, cyclopropyl, cyclobutyl, cyclopentyl, methoxy, ethoxy, and propoxy;
R³ and R⁴ are each independently selected from hydrogen, deuterium, methyl, ethyl, propyl, and isopropyl;
R⁵, R⁶, R⁷, R⁸, and R⁹ are each independently selected from hydrogen, fluorine, chlorine, bromine, iodine, methyl, ethyl, propyl, isopropyl, butyl, tert-butyl, cyclopropyl, cyclobutyl, cyclopentyl, cyclohexyl, methylthio, ethylthio, and propylthio.

Preferably, R² is selected from methyl, ethyl, n-propyl, isopropyl, cyclopropyl, cyclobutyl, and cyclopentyl.

Preferably, R³ and R⁴ are each independently selected from hydrogen, methyl, ethyl, propyl, and isopropyl.

Preferably, R⁵, R⁶, R⁷, R⁸, and R⁹ are each independently selected from hydrogen, fluorine, chlorine, bromine, iodine, cyclopropyl, cyclobutyl, cyclopentyl, cyclohexyl, methylthio, ethylthio, and propylthio.

More preferably, R² is selected from methyl, ethyl, n-propyl, isopropyl, and cyclopropyl.

More preferably, R³ and R⁴ are each independently selected from hydrogen and methyl.

More preferably, R⁵, R⁶, R⁷, R⁸, and R⁹ are each independently selected from hydrogen. chlorine, bromine, cyclopropyl, and methylthio.

In some embodiments, R⁵, R⁶, R⁸, and R⁹ are selected from hydrogen, and R⁷ is selected from methylthio.

In some embodiments, R⁵, R⁶, R⁷, and R⁹ are selected from hydrogen, and R⁸ is selected from chlorine, bromine, and methylthio.

In some embodiments, R⁵, R⁶, R⁷, and R⁸ are selected from hydrogen, and R⁹ is selected from cyclopropyl.

Furthermore, specifically, in some embodiments, the present invention provides a compound as shown in formula (III-1) and formula (III-2), and its stereoisomers, pharmaceutically acceptable salts, solvates, deuterated derivatives, metabolites, or prodrugs:

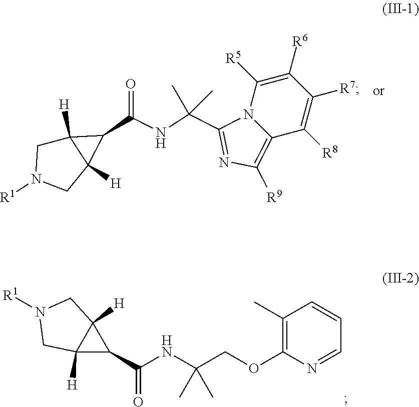

R¹ is selected from the following:

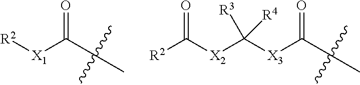

wherein,
X₁ is selected from a single bond, CH₂, or O;
X₂ and X₃ are each independently selected from CH₂ or O;
R² is selected from C1-C6 alkyl, C3-C6 cycloalkyl, substituted C1-C6 alkyl, or substituted C3-C6 cycloalkyl; substituted C1-C6 alkyl or substituted C3-C6 cycloalkyl is further substituted with 1-3 substituents independently selected from the group consisting of halogen, cyano, C1-C6 alkyl, halo-C1-C6 alkyl, C3-C6 cycloalkyl, and C1-C3 alkoxy;
R³ and R⁴ are each independently selected from hydrogen, deuterium, and C1-C3 alkyl; and
R⁵, R⁶, R⁷, R⁸, and R⁹ are each independently selected from hydrogen, halogen, cyano, C1-C6 alkyl, halo-C1-C6 alkyl, C3-C6 cycloalkyl, C1-C3 alkoxy, alkylsilyl, aryl, substituted aryl, and thioalkyl.

Further preferably, in some specific embodiments, the present invention provides a compound as shown in formula (III-1a) and formula (III-2a), or their stereoisomers, pharmaceutically acceptable salts, solvates, deuterated derivatives, metabolites, or prodrugs:

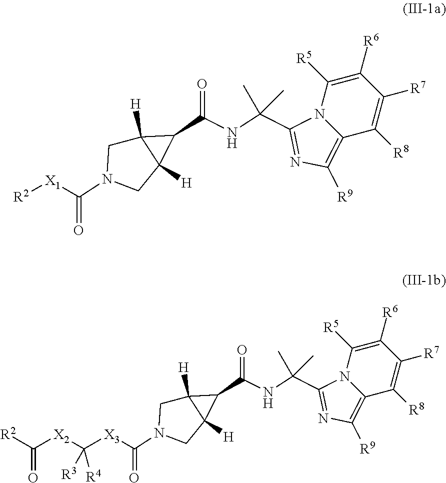

wherein:
X₁ is selected from a single bond, CH₂, or O;
X₂ and X₃ are each independently selected from CH₂ or O;
R² is selected from methyl, ethyl, propyl, isopropyl, butyl, tert-butyl, cyclopropyl, cyclobutyl, cyclopentyl, and cyclohexyl; substituted C1-C6 alkyl or substituted C3-C6 cycloalkyl is further substituted with 1-3 substituents independently selected from the group consisting of fluorine, chlorine, bromine, iodine, cyano, methyl, ethyl, propyl, fluoromethyl, difluoromethyl, trifluoromethyl, cyclopropyl, cyclobutyl, cyclopentyl, methoxy, ethoxy, and propoxy;
R³ and R⁴ are each independently selected from hydrogen, deuterium, methyl, ethyl, propyl, and isopropyl; and
R⁵, R⁶, R⁷, R⁸, and R⁹ are each independently selected from hydrogen, fluorine, chlorine, bromine, iodine, methyl, ethyl, propyl, isopropyl, butyl, tert-butyl, cyclopropyl, cyclobutyl, cyclopentyl, cyclohexyl, methylthio, ethylthio, and propylthio.

Preferably, X₁ is selected from a single bond or CH₂.

Preferably, X₂ and X₃ are each independently selected from O.

Preferably, R² is selected from methyl, ethyl, n-propyl, isopropyl, cyclopropyl, cyclobutyl, and cyclopentyl.

Preferably, R³ and R⁴ are each independently selected from hydrogen, methyl, ethyl, propyl, and isopropyl.

Preferably, R⁵, R⁶, R⁷, R⁸, and R⁹ are each independently selected from hydrogen, fluorine, chlorine, bromine, iodine, cyclopropyl, cyclobutyl, cyclopentyl, cyclohexyl, methylthio, ethylthio, and propylthio.

More preferably, R² is selected from methyl, ethyl, n-propyl, isopropyl, and cyclopropyl.

More preferably, R³ and R⁴ are each independently selected from hydrogen, methyl, and ethyl.

More preferably, R⁵, R⁶, R⁷, R⁸, and R⁹ are each independently selected from hydrogen, fluorine, chlorine, bromine, iodine, cyclopropyl, and methylthio.

Further preferably, R² is selected from isopropyl and cyclopropyl.

Further preferably, R³ and R⁴ are each independently selected from hydrogen and methyl.

Further preferably, R⁵, R⁶, R⁷, R⁸, and R⁹ are each independently selected from hydrogen, chlorine, bromine, cyclopropyl, and methylthio.

In some embodiments, R⁵, R⁶, R⁸, and R⁹ are selected from hydrogen, and R⁷ is selected from methylthio.

In some embodiments, R⁵, R⁶, R⁷, and R⁹ are selected from hydrogen, and R⁸ is selected from chlorine, bromine, and methylthio.

In some embodiments, R⁵, R⁶, R⁷, and R⁸ are selected from hydrogen, and R⁹ is selected from cyclopropyl.

Furthermore, the compound is selected from, but not limited to, the following compounds and their stereoisomers, pharmaceutically acceptable salts, solvates, deuterated derivatives, metabolites, or prodrugs:

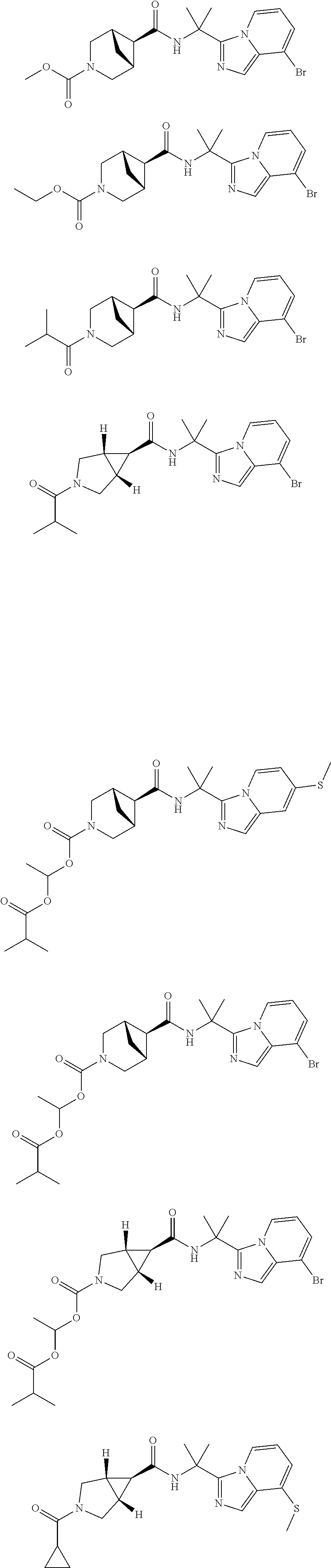

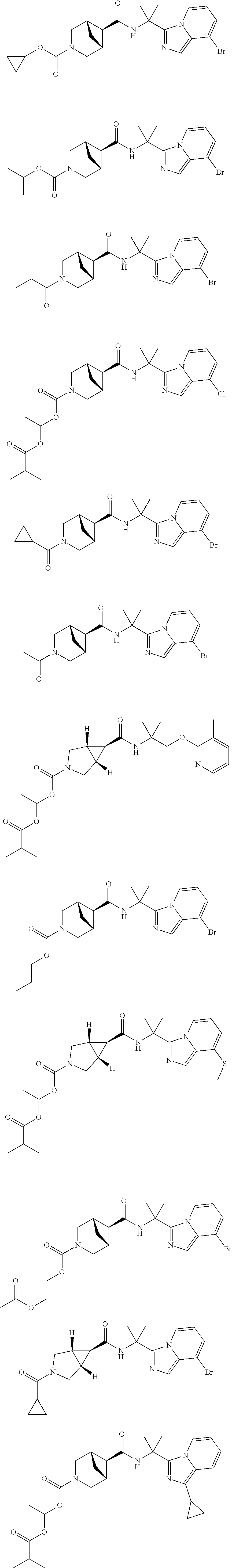

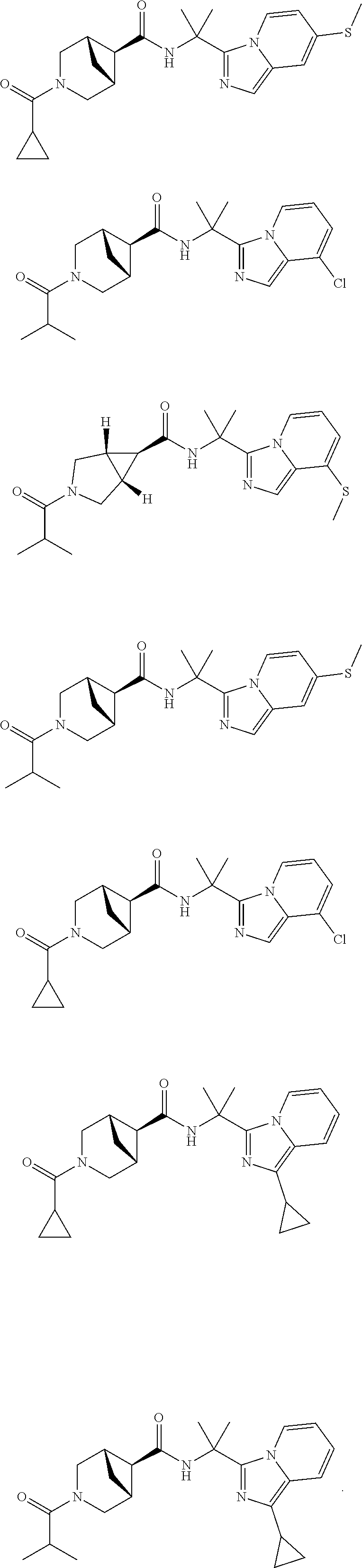

In another aspect, the present invention provides a pharmaceutical composition comprising a compound of formula (I) and its stereoisomers, pharmaceutically acceptable salts, solvates, deuterated derivatives, metabolites, or prodrugs, along with at least one pharmaceutically acceptable excipient.

In a third aspect, the present invention provides a method for synthesizing a compound of formula (I), which includes the following step: Condensing a compound of formula (I-1) with a compound of formula (I-2) under basic conditions to obtain a compound of formula (I).

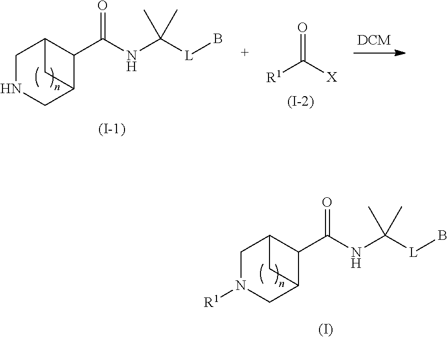

wherein, X is a leaving group such as chlorine, hydroxyl, p-nitrophenyl, etc.; the basic conditions involve adding an organic base such as TEA, DIPEA, pyridine, etc.

In a fourth aspect, the present invention provides a method for treating SSTR4-related diseases or disorders. This method includes administering to an individual in need thereof a composition comprising a compound of formula (I).

### Beneficial Effects

The compounds of the present invention are prodrugs of small molecule SSTR4 agonists, which can improve their pharmacokinetics, have good oral bioavailability, and can reduce side effects, making them suitable for medical use and of clinical value. The compounds and their pharmaceutical compositions can be used to prepare medications for the treatment and/or prevention of pain conditions associated with the SSTR4 receptor. Additionally, the synthesis steps of the compounds in the present invention are simple, thus providing good economic value.

### Definition and Explanation of Terms

Unless otherwise specified, the groups and terms defined in the specification and claims of this application, including those described as examples, exemplary, preferred, in tables, and as specific compounds in the embodiments, can be combined and interchanged in any manner. Subsequent group definitions and compound structures should be considered within the scope of the specification.

The compounds described herein may have asymmetric centers. Compounds of the invention containing asymmetric substituted atoms can be isolated in optically active or racemic forms. Unless specific stereochemistry or isomeric forms are indicated, all chiral, diastereomeric, racemic forms, and all geometric isomeric forms of structures are applicable.

As used herein, the term “alkyl” refers to straight or branched chain saturated aliphatic hydrocarbon groups having from 1 to 20 carbon atoms, preferably from 1 to 8 carbon atoms, more preferably from 1 to 6 carbon atoms, and further preferably from 1 to 4 carbon atoms. Non-limiting examples include methyl, ethyl, n-propyl, isopropyl, n-butyl, sec-butyl, isobutyl, tert-butyl, n-pentyl, isopentyl, neopentyl, n-hexyl, and various branched isomers thereof; the groups may be optionally further substituted with 0 to 6 substituents independently selected from F, Cl, Br, I, hydroxyl, mercapto, nitro, cyano, amino, alkylamino, amido, alkenyl, alkynyl, C1-6 alkyl, C1-6 hydroxyalkyl, C1-6 alkoxy, 3- to 8-membered carbocyclic, 3- to 8-membered heterocyclic, 3- to 8-membered carbocyclic-oxy, 3- to 8-membered heterocyclic-oxy, carboxyl, or carboxylic ester groups. The term “alkyl” as used herein is consistent with this definition.

The term “C3-C6 cycloalkyl” as used herein is to be understood as representing saturated monovalent monocyclic or bicyclic hydrocarbon rings having 3 to 6 carbon atoms, including fused or bridged polycyclic systems, such as cyclopropyl, cyclobutyl, cyclopentyl, and cyclohexyl. The C3-C6 cycloalkyl group may optionally be further substituted with 0 to 6 substituents independently selected from F, Cl, Br, I, ═O, hydroxyl, mercapto, nitro, cyano, amino, alkylamino, amido, alkenyl, alkynyl, alkyl, hydroxyalkyl, alkoxy, carbocyclic, heterocyclic, carbocyclic-oxy, heterocyclic-oxy, carboxyl, or carboxylic ester groups. The term “C3-C6 cycloalkyl” as used herein is consistent with this definition.

The term “alkoxy” as used herein refers to—O-alkyl. Non-limiting examples include methoxy, ethoxy, n-propoxy, isopropoxy, n-butoxy, sec-butoxy, tert-butoxy, n-pentoxy, n-hexyloxy, cyclopropoxy, and cyclobutoxy. The alkyl group may optionally be further substituted with 0 to 6 substituents independently selected from F, Cl, Br, I, hydroxyl, mercapto, nitro, cyano, amino, alkylamino, alkenyl, alkynyl, alkyl, hydroxyalkyl, alkoxy, carbocyclic, heterocyclic, carbocyclic-oxy, heterocyclic-oxy, carboxyl, or carboxylic ester groups. The term “alkoxy” as used herein is consistent with this definition.

The term “thioalkyl” as used herein refers to—S-alkyl. Non-limiting examples include methylthio, ethylthio, n-propylthio, isopropylthio, n-butylthio, sec-butylthio, tert-butylthio, n-pentylthio, n-hexylthio, cyclopropylthio, and cyclobutylthio. The alkyl group may optionally be further substituted with 0 to 5 substituents independently selected from F, Cl, Br, I, hydroxyl, mercapto, nitro, cyano, amino, alkylamino, alkenyl, alkynyl, alkyl, hydroxyalkyl, alkoxy, carbocyclic, heterocyclic, carbocyclic-oxy, heterocyclic-oxy, carboxyl, or carboxylic ester groups. The term “thioalkyl” as used herein is consistent with this definition.

The term “aryl” used alone or as part of another group refers to optionally substituted homocyclic or heterocyclic conjugated planar ring systems containing delocalized electrons. These aryl groups preferably contain 5-14 ring atoms in the ring portion, including monocyclic (e.g., furan or benzene), bicyclic, or tricyclic groups. The term “aromatic” includes “aryl” as defined below.

The term “aryl” or “Ar” used alone or as part of another group refers to optionally substituted homocyclic aromatic groups, preferably monocyclic or bicyclic groups containing 6 to 10 carbon atoms in the ring portion, such as phenyl, biphenyl, naphthyl, substituted phenyl, substituted biphenyl, or substituted naphthyl.

The term “carbocyclic” or “carbocyclic” used alone or as part of another group refers to optionally substituted, aromatic or non-aromatic homocyclic rings or ring systems, in which all atoms in the ring are carbon atoms, preferably having 5 or 6 carbon atoms in each ring. Exemplary substituents include one or more of the following groups: hydrocarbon, substituted hydrocarbon, alkyl, alkoxy, acyl, acyloxy, alkenyl, alkenyloxy, aryl, aryloxy, amino, amido, acetal, aminocarbonyl, carbocyclic, cyano, ester, ether, halogen, heterocyclic, hydroxy, keto, ketal, phospho, nitro, and thio groups.

The term “heteroaryl” used alone or as part of another group refers to optionally substituted aromatic groups having at least one heteroatom in at least one ring, preferably having 5 or 6 ring atoms in each ring. Heteroaryl groups preferably contain 1 or 2 oxygen atoms and/or 1 to 4 nitrogen atoms in the ring and are bonded to the rest of the molecule through carbon. Exemplary groups include furanyl, benzofuranyl, oxazolyl, isoxazolyl, oxadiazolyl, benzoxazolyl, benzoxadiazolyl, pyrrolyl, pyrazolyl, imidazolyl, triazolyl, tetrazolyl, pyridyl, pyrimidyl, pyrazinyl, pyridazinyl, indolyl, isoindolyl, indolizinyl, benzimidazolyl, indazolyl, benzotriazolyl, tetrazolopyrazinyl, carbazolyl, purinyl, quinolinyl, isoquinolinyl, imidazopyridyl, and the like. Exemplary substituents include one or more of the following groups: hydrocarbon, substituted hydrocarbon, alkyl, alkoxy, acyl, acyloxy, alkenyl, alkenyloxy, aryl, aryloxy, amino, amido, acetal, aminocarbonyl, carbocyclic, cyano, ester, ether, halogen, heterocyclic, hydroxy, keto, ketal, phospho, nitro, and thio groups.

The term “heterocyclic” or “heterocyclic” used alone or as part of another group refers to optionally substituted, fully saturated or unsaturated, monocyclic or bicyclic, aromatic or non-aromatic groups having at least one heteroatom in at least one ring, preferably having 5 or 6 ring atoms in each ring. Heterocyclic groups preferably contain 1 or 2 oxygen atoms and/or 1 to 4 nitrogen atoms in the ring and are bonded to the rest of the molecule through carbon or heteroatoms. Exemplary heterocyclic groups include the heteroaryl compounds described above. Exemplary substituents include one or more of the following groups: hydrocarbon, substituted hydrocarbon, alkyl, alkoxy, acyl, acyloxy, alkenyl, alkenyloxy, aryl, aryloxy, amino, amido, acetal, aminocarbonyl, carbocyclic, cyano, ester, ether, halogen, heterocyclic, hydroxy, keto, ketal, phospho, nitro, and thio groups.

The term “protecting group” used herein refers to groups that can protect specific parts of a molecule, which can be removed after the protected reaction without interfering with the rest of the molecule. When the part is an oxygen atom (forming a protected hydroxyl), exemplary protecting groups include ethers (e.g., allyl, triphenylmethyl (trityl or Tr), benzyl, p-methoxybenzyl (PMB), p-methylphenyl (PMP)), acetals (e.g., methoxymethyl (MOM), β-methoxyethoxymethyl (MEM), tetrahydropyranyl (THP), ethoxyethyl (EE), methylthiomethyl (MTM), 2-methoxy-2-propyl (MOP), 2-(trimethylsilyl) ethoxymethyl (SEM)), esters (e.g., benzoates (Bz), carbonates (e.g., allyl carbonate, 2,2,2-trichloroethyl carbonate (Troc), t-butyldimethylsilyl (TBDMS), t-butyldiphenylsilyl (TBDPS)), and silyl ethers (e.g., trimethylsilyl (TMS), triethylsilyl (TES), triisopropylsilyl (TIPS), triphenylsilyl (TPS), t-butyldimethylsilyl (TBDMS), t-butyldiphenylsilyl (TBDPS)). When the part is a nitrogen atom (forming a protected amine), exemplary protecting groups include benzyl (e.g., p-methoxyphenyl (PMP), 3,4-dimethoxyphenoxy (PMB)), esters (e.g., benzoates (Bz)), carbonyl (e.g., p-methoxybenzyloxycarbonyl (Moz), t-butoxycarbonyl (BOC), 9-fluorenylmethoxycarbonyl (FMOC)), acetyl, carbamates, and N-silyl groups. Various protecting groups and their synthetic methods can be found in P. G. M. Wuts and T. W. Greene's “Greene's Protective Groups in Organic Synthesis” (4th Edition), John Wiley & Sons, Inc.

The term “substituted hydrocarbon” refers to a hydrocarbon group substituted with at least one non-carbon atom, including portions where the carbon chain atoms are replaced by heteroatoms, such as nitrogen, oxygen, silicon, phosphorus, boron, or halogen atoms, as well as portions where the carbon chain contains additional substituents. These substituents include alkyl, alkoxy, acyl, acyloxy, alkenyl, alkenyloxy, aryl, aryloxy, amino, amido, acetal, aminocarbonyl, carbocyclic, cyano, ester, ether, halogen, heterocyclic, hydroxy, keto, ketal, phospho, nitro, and thio groups.

The terms “comprising,” “including,” and “having” are meant to be inclusive, indicating that elements other than those listed can be present. Modifications and variations can be made to the invention without departing from the scope defined by the appended claims.

Other features and advantages of the invention will become apparent from the following description and will be understood in part through the implementation of the invention. The objectives and other advantages of the invention can be realized and obtained by the structures particularly pointed out in the written description, claims, and drawings.

### EXAMPLES

The general formula compounds of the present invention, as well as their preparation methods and uses, will be further detailed below in conjunction with specific embodiments. The following examples are for illustrative purposes and to explain the invention and should not be construed as limiting the scope of protection of the invention. Any technology realized based on the above content of the invention is encompassed within the intended scope of protection of the invention.

Unless otherwise specified, the raw materials and reagents used in the following examples are commercially available or can be prepared by known methods.

This application uses the following abbreviations: ACN: Acetonitrile, DIPEA: N,N-Diisopropylethylamine, DCM: dichloromethane, and TEA: Triethylamine.

Compounds are named according to conventional nomenclature rules in the field; commercially available reagents are referred to by their catalog names from suppliers.

¹H NMR data were collected and recorded using a Bruker Avance Neo 400 MHz superconducting liquid NMR spectrometer at 400 MHz, using DMSO-d6, MeOD, and Chloroform-d as solvents, and TMS (δ=0) as the internal standard to report chemical shift δ values (ppm). Mass spectra were collected and recorded using a Waters ACQUITY UPLC with an ACQUITY UPLC BEH C8, 50 mm×2.1 mm×1.7 um (20180306-C8-08) column. Mobile phase A: 0.01% TFA/H₂O; mobile phase B: CH₃CN; flow rate: 0.2 mL/min; column temperature: 30° C.; detection wavelength: UV-210 nm. High-performance liquid chromatography (HPLC) was measured using a Thermo UltiMate 3000 liquid chromatograph with a Venusil ASB C18 (4.6250 mm, 5 um) column. Mobile phase A: phosphoric acid aqueous solution with pH=1.5; mobile phase B: CH₃CN; flow rate: 1.0 mL/min; column temperature: 35° C.; detection wavelength: UV-215 nm; injection volume: 2 μL; gradient elution conditions: elution at a flow rate of 1.0 mL/min throughout, first with 95% A and 5% B for 10 min, then with 20% A and 80% B for 5 min, and finally with 95% A and 5% B for 5 min. Percentages represent the volume percentage of the mobile phase in the elution solution.

### Example 1: Synthesis of Compound 1

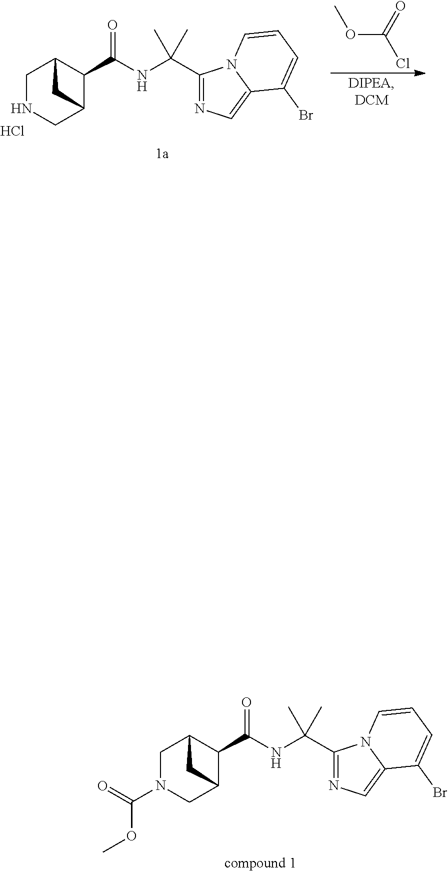

A mixture of compound 1a (80.0 mg, 193 μmol, 1.00 eq, HCl), DIPEA (50.0 mg, 387 μmol, 67.36 μL, 2.00 eq) in DCM (1.00 mL) was degassed and purged with N₂ for 3 times, and then added methyl carbonochloridate (21.9 mg, 232 μmol, 17.9 μL, 1.20 eq) at 0° C., the mixture was stirred at 0° C. for 1 hr under N₂ atmosphere. LCMS showed compound 1a was consumed completely and desired peak (Rt=0.298 min, MS=435.0, M+H+) was detected. The reaction mixture was quenched by 5.00 ml of ice water, then extracted with ethyl acetate (5.00 mL×3), washed with brine (5.00 mL×2), filtered and the filtrate was concentrated under reduced pressure. The crude product was purified by reversed-phase HPLC (column: Phenomenex luna C18 150×25 mm×10 um; mobile phase: [water (HCl)-ACN]; gradient: 8%-38% B over 10 min) to obtain compound 1 (72.0 mg, 165 μmol, 85.5% yield, 100% purity) as a white solid.

LCMS: Rt=0.324 min, MS=435.1, M+H⁺, 5-95AB_0.8 min

¹HNMR: 400 MHZ, DMSO-d₆

δ 8.71 (s, 1H), 8.43 (d, J=7.2 Hz, 1H), 7.83 (s, 1H), 7.33 (d, J=6.8 Hz, 1H), 6.81 (t, J=7.2 Hz, 1H), 3.61 (s, 3H), 3.45-3.60 (m, 5H), 2.40-2.47 (m, 2H), 1.97-2.07 (m, 1H), 1.73 (s, 6H), 1.09-1.16 (m, 1H).

### Example 2: Synthesis of Compound 2

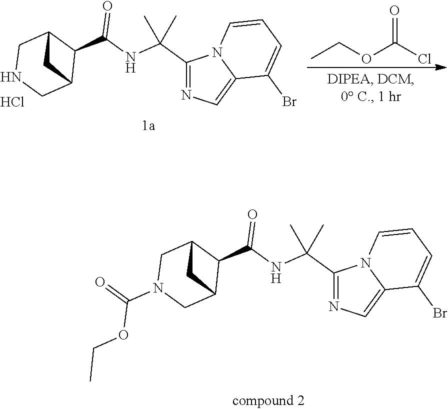

A mixture of compound 1a (80.0 mg, 193 μmol, 1.00 eq, HCl), DIPEA (50.0 mg, 387 μmol, 67.4 μL, 2.00 eq) in DCM (1.00 mL) was degassed and purged with N₂ for 3 times, and then added ethyl carbonochloridate (25.2 mg, 232 μmol, 22.2 μL, 1.20 eq) at 0° C., the mixture was stirred at 0° C. for 1 hr under N₂ atmosphere. LCMS showed compound 1a was consumed completely and desired peak (Rt=0.320 min, MS=449.0, M+H⁺) was detected. The reaction mixture was quenched by 5.00 mL of ice water, then extracted with ethyl acetate (5.00 mL×3), washed with brine (5.00 mL×2), filtered and the filtrate was concentrated under reduced pressure. The crude product was purified by reversed-phase HPLC (column: Phenomenex luna C18 150×25 mm×10 μm; mobile phase: [water (HCl)-ACN]; gradient: 8%-38% B over 10 min) to obtain compound 2 (66.0 mg, 147 μmol, 76.0% yield, 100% purity) as a white solid.

LCMS: Rt=0.347 min, MS=449.0, M+H⁺, 5-95AB_0.8 min 1HNMR: 400 MHZ, DMSO-d₆,

δ 8.77 (s, 1H), 8.46 (d, J=7.2 Hz, 1H), 7.88 (s, 1H), 7.36 (d, J=6.8 Hz, 1H), 6.84 (t, J=7.2 Hz, 1H), 4.00-4.10 (m, 3H), 3.43-3.62 (m, 4H), 2.42-2.48 (m, 2H), 1.97-2.09 (m, 1H), 1.74 (s, 6H), 1.19 (t, J=7.2 Hz, 3H), 1.08-1.15 (m, 1H).

### Example 3: Synthesis of Compound 3

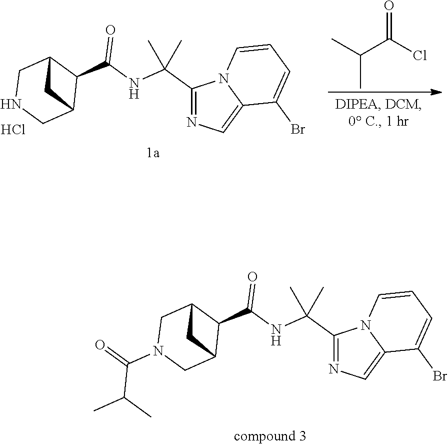

A mixture of compound 1a (80.0 mg, 193 μmol, 1.00 eq, HCl), DIPEA (50.0 mg, 387 μmol, 67.4 μL, 2.00 eq) in DCM (1.00 mL) was degassed and purged with N₂ for 3 times, and then added ethyl 2-methylpropanoyl chloride (24.7 mg, 232 μmol, 24.3 μL, 1.20 eq) at 0° C., the mixture was stirred at 0° C. for 1 hr under N₂ atmosphere. LCMS showed compound 1a was consumed completely and desired peak (Rt=0.327 min, MS=447.1, M+H⁺) was detected. The reaction mixture was quenched by 5.00 mL of ice water, then extracted with ethyl acetate (5.00 mL×3), washed with brine (5.00 mL×2), filtered and the filtrate was concentrated under reduced pressure. The crude product was purified by reversed-phase HPLC (column: Phenomenex luna C18 150×25 mm×10 μm; mobile phase: [water (HCl)-ACN]; gradient: 8%-38% B over 10 min) to obtain compound 3 (85.0 mg, 189 μmol, 97.6% yield, 99.3% purity) as a white solid.

LCMS: Rt=0.299 min, MS=447.0, M+H⁺, 5-95AB_0.8 min

¹HNMR: 400 MHZ, DMSO-d₆

δ 8.69 (s, 1H), 8.43 (d, J=7.2 Hz, 1H), 7.83 (s, 1H), 7.33 (d, J=6.8 Hz, 1H), 6.81 (t, J=7.2 Hz, 1H), 3.70-3.82 (m, 3H), 3.45-3.60 (m, 3H), 2.75-2.85 (m, 1H), 2.42-2.45 (m, 1H), 2.01-2.09 (m, 1H), 1.73 (d, J=3.6 Hz, 6H), 1.08-1.15 (m, 1H), 1.02 (t, J=7.2 Hz, 6H).

### Example 4: Synthesis of Compound 4

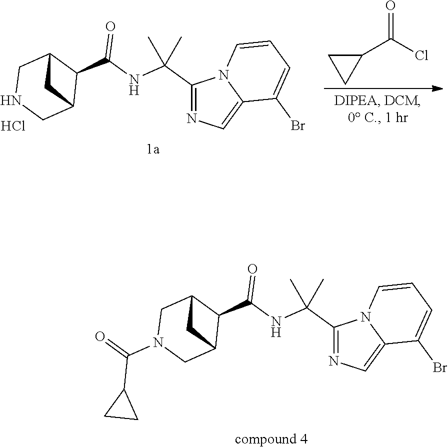

A mixture of compound 1a (80.0 mg, 193 μmol, 1.00 eq, HCl), DIPEA (50.0 mg, 387 μmol, 67.4 μL, 2.00 eq) in DCM (1.00 mL) was degassed and purged with N₂ for 3 times, and then added cyclopropanecarbonyl chloride (24.3 mg, 232 μmol, 21.1 μL, 1.20 eq) at 0° C., the mixture was stirred at 0° C. for 1 hr under N₂ atmosphere. LCMS showed compound 1a was consumed completely and desired peak (Rt=0.320 min, MS=445.1, M+H⁺) was detected. The reaction mixture was quenched by 5.00 mL of ice water, then extracted with ethyl acetate (5.00 mL×3), washed with brine (5.00 mL×2), filtered and the filtrate was concentrated under reduced pressure. The crude product was purified by reversed-phase HPLC (column: Phenomenex luna C18 150*25 mm*10 um; mobile phase: [water (HCl)-ACN]; gradient: 8%-38% B over 10 min) to obtain compound 4 (82.0 mg, 184 μmol, 95.2% yield, 100% purity) as a white solid.

LCMS: Rt=0.290 min, MS=445.1, M+H⁺, 5-95AB_0.8 min

¹HNMR: 400 MHZ, DMSO-d₆

δ 8.80 (s, 1H), 8.47 (d, J=7.2 Hz, 1H), 7.91 (s, 1H), 7.38 (d, J=6.8 Hz, 1H), 6.86 (t, J=7.2 Hz, 1H), 3.85-3.96 (s, 3H), 3.45-3.63 (m, 3H), 2.45-2.48 (m, 1H), 2.01-2.09 (m, 1H), 1.82-1.90 (m, 1H), 1.75 (s, 6H), 1.08-1.15 (m, 1H), 0.70-0.79 (s, 4H).

### Example 5: Synthesis of Compound 5

### Step 1: Synthesis of Intermediate 5a

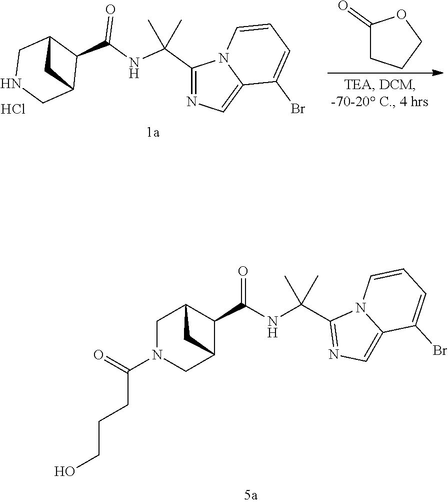 [SMILES: Cl ; CC(C)(NC(=O)[C@H]1[C@@H]2CNC[C@H]1C2)c1ncc2c(Br)cccn12 ; O=C1CCCO1 ; CC(C)(NC(=O)[C@@H]1[C@@H]2C[C@H]1CN(C(=O)CCCO)C2)c1ncc2c(Br)cccn12]

A mixture of compound 1a (100 mg, 242 μmol, 1.00 eq, HCl), TEA (73.4 mg, 725 μmol, 100.92 μL, 3.00 eq) in DCM (1.00 mL) was degassed and purged with N₂ for 3 times, and then added tetrahydrofuran-2-one (20.8 mg, 242 μmol, 18.44 μL, 1.00 eq) at −70° C., the mixture was stirred at 20° C. for 4 hrs under N₂ atmosphere. LCMS showed compound 1a was consumed completely and desired peak (Rt=0.329 min, MS=463.1, M+H⁺) was detected. The reaction mixture was quenched by 5.00 mL of ice water, then extracted with ethyl acetate (5.00 mL×3), washed with brine (5.00 mL×2), filtered and the filtrate was concentrated under reduced pressure to obtain compound 5a (268 mg, crude) as a brown gum.

LCMS: Rt=0.329 min, MS=463.1, M+H⁺

### Step 1: Synthesis of Intermediate 5

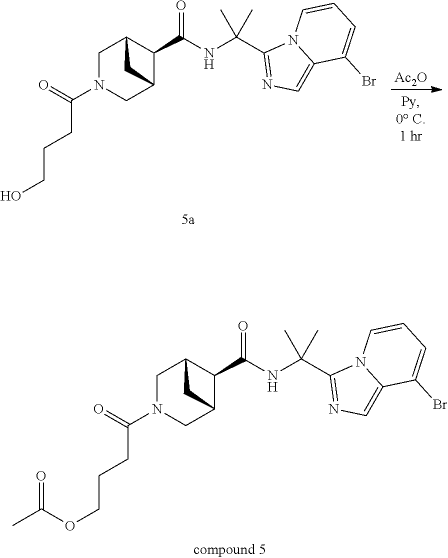 [SMILES: "Ac2O" ; CC(C)(NC(=O)[C@@H]1[C@@H]2C[C@H]1CN(C(=O)CCCO)C2)c1ncc2c(Br)cccn12 ; CC(=O)OCCCC(=O)N1C[C@H]2C[C@@H](C1)[C@@H]2C(=O)NC(C)(C)c1ncc2c(Br)cccn12]

A mixture of compound 5a (128 mg, 276 μmol, 1.00 eq), Py (65.6 mg, 829 μmol, 66.9 μL, 3.00 eq) in Ac₂O (1.00 mL) was degassed and purged with N₂ for 3 times, the mixture was stirred at 0° C. for 1 hr under N₂ atmosphere. TLC (dichloromethane:methanol=10:1) indicated compound 5a (R_{f}=0.31) was consumed completely and one new spot (R_{f}=0.58) formed. The crude product was purified by reversed-phase HPLC (column: CD02-Waters Xbidge BEH C18 150× 25 mm×10 μm; mobile phase: [water (NH₄HCO₃)-ACN]; gradient: 16%-46% B over 10 min) to obtain compound 5 (58.0 mg, 110.86 μmol, 45.8% yield, 96.6% purity) as a white solid.

LCMS: Rt=0.506 min, MS=507.1, M+H⁺, 5-95AB_0.8 min

¹HNMR: 400 MHZ, MeOD

δ 8.27 (d, J=7.2 Hz, 1H), 7.40 (s, 1H), 7.03 (d, J=7.2 Hz, 1H), 6.55 (t, J=6.8 Hz, 1H), 4.13 (t, J=6.4 Hz, 2H), 3.78-3.81 (m, 2H), 3.65-3.75 (m, 2H), 2.60-2.65 (m, 2H), 2.45-2.52 (m, 3H), 2.27-2.35 (m, 1H), 2.04 (s, 3H), 1.92-2.01 (m, 2H), 1.79 (d, J=3.2 Hz, 6H), 1.20-1.30 (m, 1H).

### Example 6: Synthesis of Compound 6

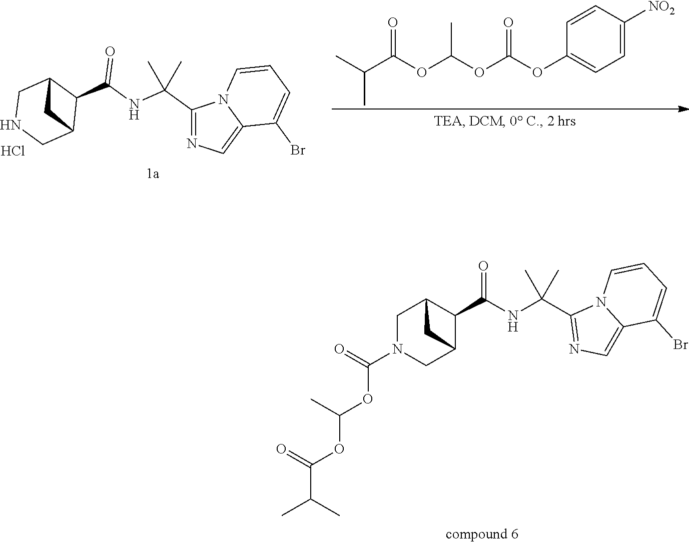 [SMILES: Cl ; CC(C)(NC(=O)[C@H]1[C@@H]2CNC[C@H]1C2)c1ncc2c(Br)cccn12 ; CC(OC(=O)Oc1ccc([N+](=O)[O-])cc1)OC(=O)C(C)C ; CC(OC(=O)C(C)C)OC(=O)N1C[C@H]2C[C@@H](C1)[C@@H]2C(=O)NC(C)(C)c1ncc2c(Br)cccn12]

To a solution of compound 1a (100 mg, 242 μmol, 1.20 eq, HCl) and 1-(4-nitrophenoxy) carbonyloxyethyl 2-methylpropanoate (60.0 mg, 202 μmol, 1.00 eq) in DCM (2.00 mL) was added TEA (61.3 mg, 606 μmol, 84.3 μL, 3.00 eq). The mixture was stirred at 0° C. for 2 hrs. LCMS showed compound 1a was consumed completely and desired peak (Rt=0.499 min, MS=535.1, M+H⁺) was detected. The reaction mixture was quenched by 5.00 mL of ice water, then extracted with ethyl acetate (5.00 mL×3), washed with brine (5.00 mL×2), filtered and the filtrate was concentrated under reduced pressure. The crude product was purified by reversed-phase HPLC (column: Phenomenex luna C18 150×25 mm×10 um; mobile phase: [water (HCl)-ACN]; gradient: 8%-38% B over 10 min) to obtain compound 6 (84.0 mg, 157 μmol, 77.7% yield, 100% purity) as a white solid.

LCMS: Rt=0.627 min, MS=535.1, M+H⁺, 5-95AB_0.8 min

¹HNMR: 400 MHZ, MeOD

δ 8.27 (d, J=7.2 Hz, 1H), 7.40 (s, 1H), 7.02 (d, J=6.8 Hz, 1H), 6.75-6.83 (m, 1H), 6.55 (t, J=7.2 Hz, 1H), 3.57-3.75 (m, 4H), 2.47-2.65 (m, 4H), 2.25-2.35 (m, 1H), 1.79 (s, 6H), 1.47-1.51 (m, 3H), 1.20-1.38 (m, 1H), 1.12-1.18 (m, 6H).

### Example 7: Synthesis of Compound 7

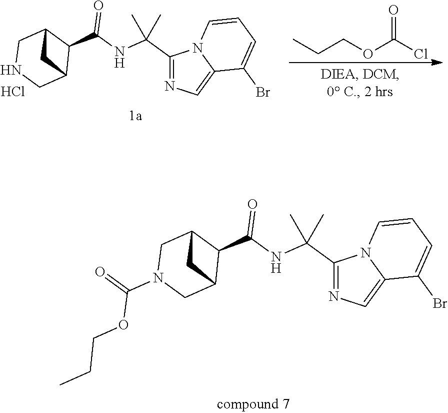

To a solution of compound 1a (80.0 mg, 193 μmol, 1.00 eq, HCl) in DCM (1.00 mL) was added DIEA (49.9 mg, 386 μmol, 67.3 μL, 2.00 eq) and propyl carbonochloridate (28.4 mg, 232 μmol, 26.0 μL, 1.20 eq) at 0° C. The mixture was stirred at 0° C. for 2 hrs. LCMS showed compound 1a was consumed completely and desired mass was detected. The reaction mixture was quenched by addition MeOH (0.50 mL) and concentrated under reduced pressure to give a residue. The residue was purified by prep-HPLC (column: Phenomenex luna C18 150×25 mm×10 μm; mobile phase: [water (HCl)-ACN]; gradient: 16%-46% B over 10 min) to give compound 7 (70.0 mg, 150 μmol, 77.9% yield, 99.7% purity) as an off-white solid.

LCMS: 5-95AB_0.8 min, Rt=0.340 min, m/z=465.1, M+H⁺

¹H NMR: 400 MHZ, MeOD

δ 8.87 (s, 1H), 8.53 (d, J=7.6 Hz, 1H), 8.14 (s, 1H), 7.54 (d, J=7.2 Hz, 2H), 7.05 (t, J=7.05 Hz, 1H), 4.04-4.089 (m, 2H), 3.62-3.68 (m, 4H), 2.53-2.58 (m, 3H), 2.23-2.25 (m, 1H), 1.90 (s, 6H), 1.65-1.70 (m, 2H), 1.23-1.24 (m, 1H), 0.97 (t, J=7.6 Hz, 3H).

### Example 8: Synthesis of Compound 8

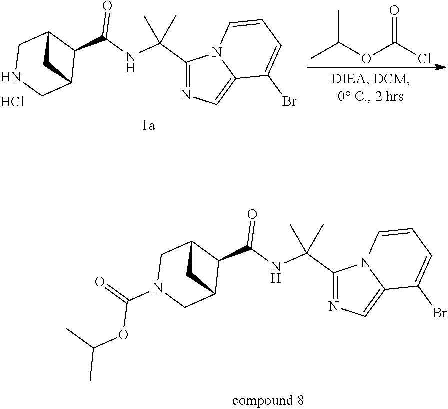

To a solution of compound 1a (80.0 mg, 193 μmol, 1.00 eq, HCl) in DCM (1.00 mL) was added DIEA (45.0 mg, 387 μmol, 67.4 μL, 2.00 eq) and isopropyl chloroformate (28.4 mg, 232 μmol, 32.2 μL, 1.20 eq) at 0° C. The mixture was stirred at 0° C. for 2 hrs. LCMS showed compound 1a was consumed completely. One new peak was shown on LCMS and desired m/z (Rt=0.334 min, MS=465.0, M+H⁺) was detected. The reaction mixture was quenched by addition MeOH (0.50 mL) and concentrated under reduced pressure to give a residue. The residue was purified by prep-HPLC (column: Phenomenex luna C18 150×25 mm×10 μm; mobile phase: [water (HCl)-ACN]; gradient: 16%-46% B over 10 min) to give compound 8 (65.0 mg, 138 μmol, 71.5% yield, 98.5% purity) as an off-white solid.

LCMS: 5-95AB_0.8 min, Rt=0.334 min, m/z=463.1, M+H⁺

¹H NMR: 400 MHZ, MeOD

δ 8.88 (s, 1H), 8.54 (d, J=7.2 Hz, 1H), 8.14 (s, 1H), 7.54 (d, J=6.8 Hz, 1H), 7.05 (t, J=6.4 Hz, 1H), 4.88-4.93 (m, 1H), 3.60-3.65 (m, 4H), 2.52-2.58 (m, 3H), 2.31-2.38 (m, 1H), 2.21-2.30 (m, 1H), 1.93 (s, 9H), 1.21-1.27 (m, 7H).

### Example 9: Synthesis of Compound 9

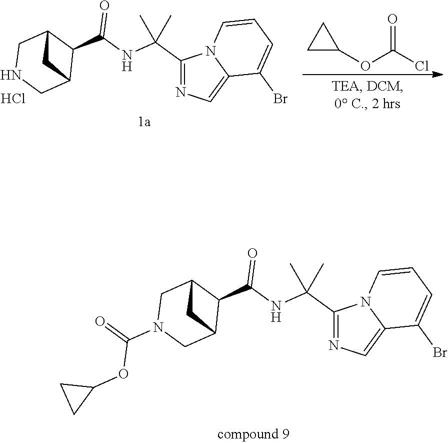

To a solution of compound 1a (80.0 mg, 193.36 μmol, 1.00 eq, HCl) in DCM (1.00 mL) was added DIPEA (49.9 mg, 386 μmol, 67.3 μL, 2.00 eq) and cyclopropyl carbonochloridate (27.9 mg, 232 μmol, 1.20 eq) at 0° C. The mixture was stirred at 0° C. for 1 hr. LCMS showed compound 1a was consumed completely and desired peak (Rt=0.316 min, MS=463.1, M+H⁺) was detected. The reaction mixture was quenched by addition MeOH (0.50 mL) and concentrated under reduced pressure to give a residue. The crude product was purified by reversed-phase HPLC (column: CD01-Phenomenex luna C18 150×25 mm×10 μm; mobile phase: [water (HCl)-ACN]; gradient: 12%-42% B over 10 min) to obtained compound 9 (70.0 mg, 151 μmol, 78.3% yield, 99.9% purity) as an off-white solid.

LCMS: Rt=0.319 min, MS=463.0, M+H⁺

¹HNMR: 400 MHZ, MeOD

δ 8.48 (d, J=7.2 Hz, 1H), 8.15 (s, 1H), 7.50 (d, J=7.2 Hz, 1H), 7.05 (t, J=7.2 Hz, 1H), 4.04-4.05 (m, 1H), 3.57-3.64 (m, 4H), 3.30-3.31 (m, 1H), 2.51-2.58 (m, 2H), 2.23-2.25 (m, 1H), 1.90 (s, 6H), 1.21-1.24 (m, 1H), 0.66-0.71 (m, 4H).

### Example 10: Synthesis of Compound 10

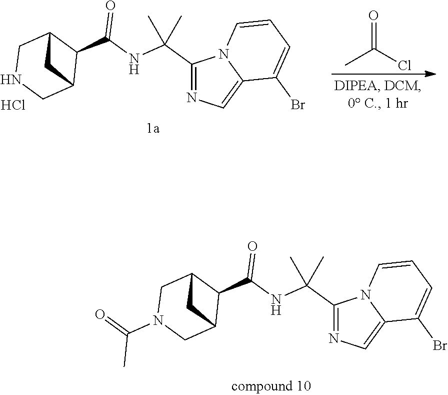

To a solution of compound 1a (80.0 mg, 193 μmol, 1.00 eq) in DCM (1.00 mL) was added DIPEA (49.9 mg, 387 μmol, 2.00 eq) and acetyl chloride (18.2 mg, 233 μmol, 1.20 eq) at 0° C. The mixture was stirred at 0° C. for 1 hr. LCMS showed compound 1a was consumed completely and one main peak with desired m/z was detected. The reaction mixture was diluted with H₂O 3.00 mL and extracted with DCM (3.00 mL×3). The combined organic layers were washed with brine (3.00 mL×2), dried over Na₂SO₄, filtered and concentrated under reduced pressure to give a residue. The residue was purified by prep-HPLC (column: Phenomenex luna C18 150×25 mm×10 μm; mobile phase: [water (FA)-ACN]; gradient: 50%-70% B over 10 min) to give compound 10 (70.0 mg, 98.5 μmol, 85.9% yield, 99.5% purity) as a white solid.

LCMS: 5-95AB_0.8 min, Rt=0.252 min, m/z=421.0, M+H⁺

¹H NMR: 400 MHZ, DMSO-d₆

δ 8.83 (s, 1H), 8.48 (d, J=2.8 Hz, 1H), 7.96 (s, 1H), 7.40 (d, J=3.2 Hz, 1H), 6.88 (t, J=2.4 Hz, 1H), 3.56 (d, J=6.4 Hz, 3H), 3.50 (d, J=4.2 Hz, 3H), 2.42 (d, J=2.8 Hz, 1H), 2.00-2.03 (m, 1H), 1.98 (s, 3H), 1.75 (s, 6H), 1.09 (d, J=3.2 Hz, 1H).

### Example 11: Synthesis of Compound 11

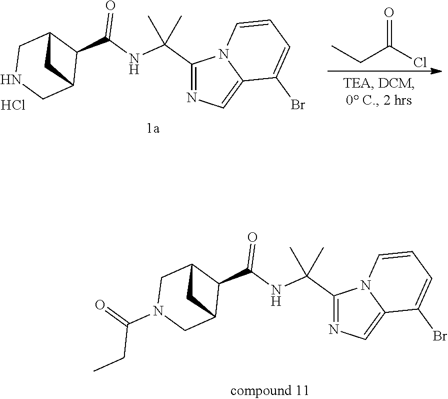

To a solution of compound 1a (80.0 mg, 193 μmol, 1.00 eq, HCl) in DCM (1.00 mL) was added TEA (49.9 mg, 386 μmol, 67.3 μL, 2.00 eq) and propionyl chloride (21.4 mg, 232 μmol, 21.4 μL, 1.20 eq) at 0° C. The mixture was stirred at 0° C. for 2 hr. LC-MS showed reactant 1a was consumed completely. One new peak was shown on LC-MS and desired m/z (Rt=0.281 min, MS=433.1, M+H⁺) was detected. The reaction mixture was quenched by addition MeOH (0.500 mL) and concentrated under reduced pressure to give a residue. The residue was purified by prep-HPLC (column: Phenomenex luna C18 150×25 mm×10 μm; mobile phase: [water (HCl)-ACN]; gradient: 6%-36% B over 10 min). Compound 11 (65.0 mg, 149 μmol, 77.4% yield, 99.8% purity) was obtained as an off-white gum.

LCMS: 5-95AB_0.8 min, Rt=0.275 min, m/z: 433.1, M+H⁺

¹H NMR: 400 MHZ, MeOD

δ 8.50 (dd, 1H), 8.20 (4, J=5.6, 8.4 Hz, 1H), 7.50 (m, 1H), 7.10 (d, J=4.4 Hz, 1H), 3.80 (m, 2H), 3.70 (dd, J=4.4, 17.1 Hz, 2H), 2.60 (dd, J=4.8, 17.1 Hz, 2H), 2.50 (m, 1H), 2.40 (m, 2H), 2.30 (s, 1H), 1.9 (s, 6H), 1.30 (s, 1H), 1.10 (s, 3H).

### Example 12: Synthesis of Compound 12

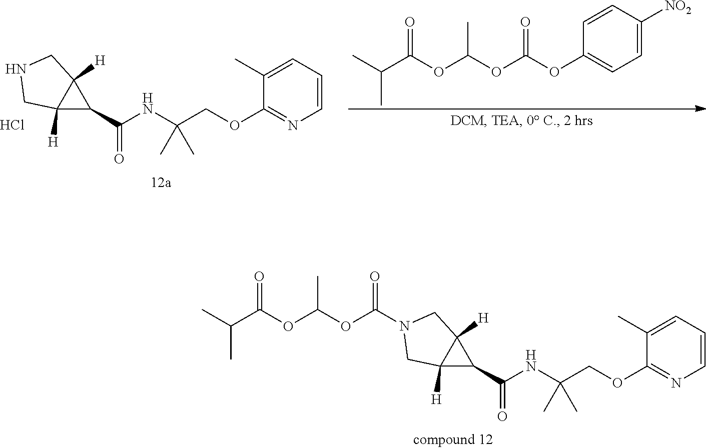

To a solution of compound 12a (100.0 mg, 307 μmol, 1.00 eq, HCl) and 1-(4-nitrophenoxy) carbonyloxyethyl 2-methylpropanoate (109.5 mg, 368 μmol, 1.20 eq) in DCM (5.00 mL) was added TEA (62.1 mg, 614 μmol, 86.2 μL, 2.00 eq) at 0° C. The mixture was stirred at 0° C. for 2 hr. LC-MS showed compound 12a was consumed completely. One new peak was shown on LC-MS and desired m/z (Rₜ=0.494 min, MS=448.3, M+H+) was detected. The reaction mixture was quenched by addition MeOH (0.500 mL) and concentrated under reduced pressure to give a residue. The residue was purified by prep-HPLC (column: Phenomenex luna C18 150× 25 mm×10 μm; mobile phase: [water (HCl)-ACN]; gradient: 6%-36% B over 10 min). Compound 12 (68.0 mg, 152 μmol, 49.5% yield, 99.9% purity) was obtained as an off-white gum.

LCMS: 5-95AB_0.8 min, Rt=0.494 min, m/z: 448.3, M+H⁺

¹H NMR: 400 MHZ, DMSO-d₆

δ 7.94 (d, J=3.1 Hz, 1H), 7.52 (d, J=7.1 Hz, 1H), 6.87 (dd, J=7.1, 5.0 Hz, 1H), 6.61 (dd, J=8.1, 5.4 Hz, 1H), 4.27 (d, J=4.1 Hz, 2H), 3.54-3.43 (m, 2H), 3.37 (dd, J=11.0, 3.8 Hz, 2H), 2.69 (s, 2H), 2.17 (s, 3H), 1.85-1.73 (m, 2H), 1.52-1.45 (m, 1H), 1.39 (dd, J=5.5, 2.4 Hz, 3H), 1.33 (s, 6H), 1.10-1.03 (m, 6H).

### Example 13: Synthesis of Compound 13

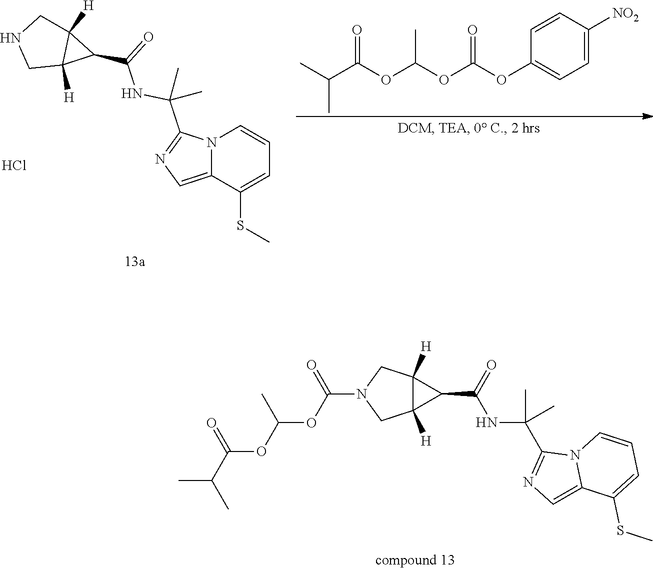 [SMILES: Cl ; CSc1cccn2c(C(C)(C)NC(=O)[C@H]3[C@@H]4CNC[C@@H]43)ncc12 ; CC(OC(=O)Oc1ccc([N+](=O)[O-])cc1)OC(=O)C(C)C ; CSc1cccn2c(C(C)(C)NC(=O)[C@H]3[C@@H]4CN(C(=O)OC(C)OC(=O)C(C)C)C[C@@H]43)ncc12]

To a solution of compound 13a (100.0 mg, 272 μmol, 1.00 eq, HCl) and 1-(4-nitrophenoxy) carbonyloxyethyl 2-methylpropanoate (97.2 mg, 327 μmol, 1.20 eq) in DCM (5.00 mL) was added TEA (55.1 mg, 545 μmol, 76.6 μL, 2.00 eq) at 0° C. The mixture was stirred at 0° C. for 1 hr. LC-MS showed 13a was consumed completely. One new peak was shown on LC-MS anddesired m/z (Rt=0.491 min, MS=489.7, M+H⁺) was detected. The reaction mixture was quenched by addition MeOH (0.500 mL) and concentrated under reduced pressure to give a residue. The residue was purified by prep-HPLC (column: Phenomenex luna C18 150×25 mm×10 μm; mobile phase: [water (HCl)-ACN]; gradient: 6%-36% B over 10 min). Compound 13 (83.0 mg, 170 μmol, 62.5% yield, 99.9% purity) was obtained as an off-white gum.

LCMS: 5-95AB_0.8 min, Rt=0.491 min, m/z: 489.7, M+H⁺

¹H NMR: 400 MHZ, Chloroform-d

δ 8.71-8.66 (m, 1H), 8.18 (s, 1H), 7.50-7.42 (m, 2H), 7.05-6.98 (m, 2H), 3.68-3.59 (m, 4H), 2.66-2.54 (m, 1H), 2.46 (s, 3H), 2.40 (t, J=6.9 Hz, 1H), 2.27-2.18 (m, 2H), 1.56 (d, J=5.3 Hz, 3H), 1.16 (d, J=7.0 Hz, 3H), 1.11 (d, J=7.0 Hz, 3H).

### Example 14: Pharmacokinetics

### Experimental Steps

SD rats (male, 6-8 weeks old, from JH Laboratory Animal Co., Ltd.) were used for intravenous injection and single oral gavage administration. Plasma samples were collected at 0.083, 0.25, 0.5, 1, 2, 4, 8, and 24 hours post-dose.

The plasma samples were obtained by centrifugation (2000 rpm, 4° C., 5 min). Samples were added to a 96-well plate and mixed with 10 volumes of acetonitrile containing an internal standard. After mixing and shaking, the mixture was centrifuged (5800 rpm, 4° C., 10 min), and the supernatant was transferred to a new 96-well plate.

The resulting solution was injected into the LC-MS/MS system. Drug concentrations in plasma were quantified using the LC-MS/MS method, and pharmacokinetic parameters were calculated accordingly.

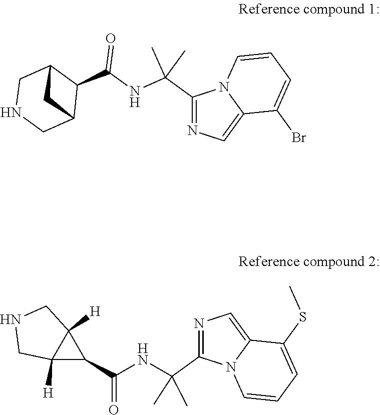 [SMILES: CC(C)(NC(=O)[C@H]1[C@@H]2CNC[C@H]1C2)c1ncc2c(Br)cccn12 ; CSc1cccn2c(C(C)(C)NC(=O)[C@H]3[C@@H]4CNC[C@@H]43)ncc12]

### Experimental Results

The pharmacokinetic parameters of the compounds in SD rats are shown in Table 1.

TABLE 1
Pharmacokinetic Parameters of the Compound in SD Rats

| | | Reference | | | Reference | | Compound |
|---|---|---|---|---|---|---|---|
| Compound | Unit | compound 1 | | Compound 6 | compound 2 | | 13 |
| Dose | mg/kg | 1 | 3 | 14.2 | 3 | 100 | 148 |
| Route | — | IV | PO | PO | IV | PO | PO |
| CL | L/hr/kg | 2.71 | — | — | 2.16 | — | — |
| Vₛₛ | L/kg | 2.38 | — | — | 6.04 | — | — |
| AUCₗₐₛₜ | hr*ng/mL | 361 | 87.3 | 877^{a} | 1410 | 13748 | 44849^{a} |
| AUC_{INF} | hr*ng/mL | 370 | 38.4 | 899^{a} | 1423 | 13834 | 45007^{a} |
| T_{1/2} | hr | 0.814 | 2.43 | 2.02^{a} | 3.99 | 3.32 | 2.96^{a} |
| MRT_{INF} | hr | 0.879 | — | — | 2.95 | — | — |
| Tₘₐₓ | hr | — | 1.42 | 0.083^{a} | — | 1.00 | 0.361^{a} |
| Cₘₐₓ | ng/mL | — | 36.3 | 602^{a} | — | 2410 | 11907^{a} |
| F | % | — | 8.06 | 24.3%^{a} | — | 29.2 | 95.8%^{a} |

^{a}After administration of the test compound, data on the parent drug in SD rats were obtained.

### Experimental Conclusion

The compounds described in this invention show advantageous pharmacokinetic properties. In prodrug form, they can be metabolized in vivo to release the parent drugs, significantly improving the bioavailability of the parent drugs. Furthermore, they have relatively high oral AUCₗₐₛₜ, which makes oral administration practical.

Numerous examples are provided herein to enhance the understanding of the present disclosure. A specific set of statements are provided as follows.

Statement 1: A compound as shown in formula (I), or its stereoisomers, pharmaceutically acceptable salts, solvates, deuterated derivatives, metabolites, or prodrugs:

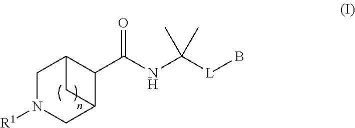

wherein:
   n=0 or 1;
   L is selected from the group consisting of a bond, —(CH₂)ₘ—, and —(CH₂)ₘO—;
wherein, m=0, 1, 2, or 3;
   B is selected from the group consisting of an aryl ring, a substituted aryl ring, a heterocyclic ring, a substituted heterocyclic ring, an alkyl-substituted heterocyclic ring, and a substituted alkyl-substituted heterocyclic ring; the B ring is further optionally substituted with 0-6 substituents independently selected from the group consisting of hydrogen, halogen, cyano, C1-C6 alkyl, halo-C1-C6 alkyl, C3-C6 cycloalkyl, C1-C3 alkoxy, alkylsilyl, aryl, substituted aryl, and thioalkyl;
   R¹ is selected from the following:

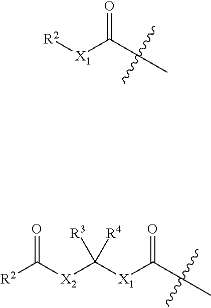

wherein:
   X₁ is selected from a single bond, CH₂, or O;
   X₂ and X₃ are each independently selected from CH₂ or O;
   R² is selected from C1-C10 alkyl, substituted C1-C10 alkyl, C3-C8 cycloalkyl, substituted C3-C8 cycloalkyl, aryl, substituted aryl, heteroaryl, substituted heteroaryl, heterocyclic, substituted heterocyclic; substituted C1-C10 alkyl, substituted C3-C8 cycloalkyl, substituted aryl, heteroaryl, or heterocyclic groups are substituted with 1-3 substituents independently selected from the group consisting of halogen, cyano, C1-C6 alkyl, halo-C1-C6 alkyl, C3-C6 cycloalkyl, and C1-C3 alkoxy; and
R³ and R⁴ are each independently selected from hydrogen, deuterium, and C1-C6 alkyl.

Statement 2: The compound of statement 1, according to formula (II) or formula (III), or its stereoisomers, pharmaceutically acceptable salts, solvates, deuterated derivatives, metabolites, or prodrugs:

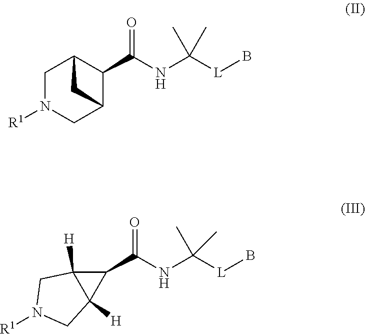

wherein:
   L is selected from the group consisting of —(CH₂)ₘ-and-(CH₂)ₘO—;
wherein m is 0 or 1;
   B is selected from the group consisting of an aryl ring, substituted aryl ring, heterocyclic ring, substituted heterocyclic ring, alkyl-substituted heterocyclic ring, and substituted alkyl-substituted heterocyclic ring; the B ring is further optionally substituted with 0-6 substituents independently selected from the group consisting of hydrogen, halogen, cyano, C1-C6 alkyl, halo-C1-C6 alkyl, C3-C6 cycloalkyl, C1-C3 alkoxy, alkylsilyl, aryl, substituted aryl, and thioalkyl;
   R¹ is selected from the following:

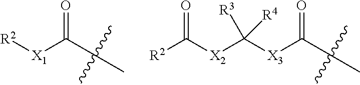

wherein:
   X₁ is selected from a single bond, CH₂, or O;
   X₂ and X₃ are each independently selected from CH₂ or O;
   R² is selected from C1-C10 alkyl, C3-C8 cycloalkyl, substituted C1-C10 alkyl, or substituted C3-C8 cycloalkyl; substituted C1-C10 alkyl or substituted C3-C8 cycloalkyl is further substituted with 1-3 substituents independently selected from the group consisting of halogen, cyano, C1-C6 alkyl, halo-C1-C6 alkyl, C3-C6 cycloalkyl, and C1-C3 alkoxy; and
   R³ and R⁴ are each independently selected from hydrogen, deuterium, and C1-C3 alkyl.

Statement 3: The compound of statement 2, wherein B is selected from phenyl, naphthyl, pyridyl, pyrimidyl, and imidazopyridyl.

Statement 4: The compound of any one of statements 1-3, according to formula (II-1), or its stereoisomers, pharmaceutically acceptable salts, solvates, deuterated derivatives, metabolites, or prodrugs:

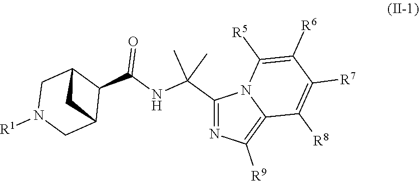

R¹ is selected from the following:

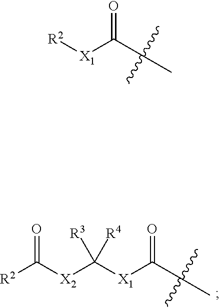

wherein:
X₁ is selected from a single bond, CH₂, or O;
X₂ and X₃ are each independently selected from CH₂ or O;
R² is selected from C1-C6 alkyl, C3-C6 cycloalkyl, substituted C1-C6 alkyl, or substituted C3-C6 cycloalkyl; substituted C1-C6 alkyl or substituted C3-C6 cycloalkyl is further substituted with 1-3 substituents independently selected from the group consisting of halogen, cyano, C1-C6 alkyl, halo-C1-C6 alkyl, C3-C6 cycloalkyl, and C1-C3 alkoxy;
R³ and R⁴ are each independently selected from hydrogen, deuterium, and C1-C3 alkyl; and
R⁵, R⁶, R⁷, R⁸, and R⁹ are each independently selected from hydrogen, halogen, cyano, C1-C6 alkyl, halo-C1-C6 alkyl, C3-C6 cycloalkyl, C1-C3 alkoxy, alkylsilyl, aryl, substituted aryl, and thioalkyl.

Statement 5: The compound of statement 1 or statement 4, according to formula (II-1a) or formula (II-1b), or its stereoisomers, pharmaceutically acceptable salts, solvates, deuterated derivatives, metabolites, or prodrugs:

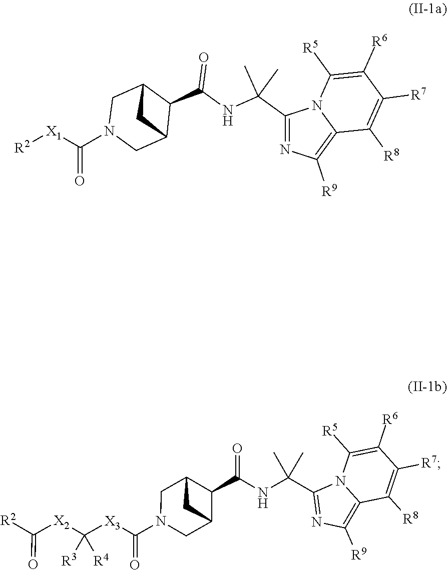

R¹ is selected from the following:

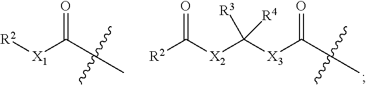

wherein,
   X₁ is selected from a single bond, CH₂, or O;
   X₂ and X₃ are each independently selected from CH₂ or O;
   R² is selected from methyl, ethyl, propyl, isopropyl, butyl, tert-butyl, cyclopropyl, cyclobutyl, cyclopentyl, cyclohexyl; substituted C1-C6 alkyl or substituted C3-C6 cycloalkyl is further substituted with 1-3 substituents independently selected from the group consisting of fluorine, chlorine, bromine, iodine, cyano, methyl, ethyl, propyl, fluoromethyl, difluoromethyl, trifluoromethyl, cyclopropyl, cyclobutyl, cyclopentyl, methoxy, ethoxy, and propoxy;
   R³ and R⁴ are each independently selected from hydrogen, deuterium, methyl, ethyl, propyl, and isopropyl;
   R⁵, R⁶, R⁷, R⁸, and R⁹ are each independently selected from hydrogen, fluorine, chlorine, bromine, iodine, methyl, ethyl, propyl, isopropyl, butyl, tert-butyl, cyclopropyl, cyclobutyl, cyclopentyl, cyclohexyl, methylthio, ethylthio, and propylthio.

Statement 6: The compound of statement 1 or 2, according to formula (III-1) or formula (III-2), or its stereoisomers, pharmaceutically acceptable salts, solvates, deuterated derivatives, metabolites, or prodrugs:

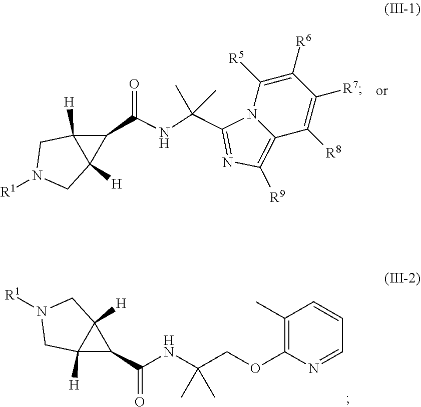

R¹ is selected from the following:

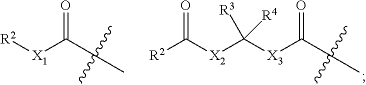

wherein:
   X₁ is selected from a single bond, CH₂, or O;
   X₂ and X₃ are each independently selected from CH₂ or O;
   R² is selected from C1-C6 alkyl, C3-C6 cycloalkyl, substituted C1-C6 alkyl, or substituted C3-C6 cycloalkyl; substituted C1-C6 alkyl or substituted C3-C6 cycloalkyl is further substituted with 1-3 substituents independently selected from the group consisting of halogen, cyano, C1-C6 alkyl, halo-C1-C6 alkyl, C3-C6 cycloalkyl, and C1-C3 alkoxy;
   R³ and R⁴ are each independently selected from hydrogen, deuterium, and C1-C3 alkyl; and
   R⁵, R⁶, R⁷, R⁸, and R⁹ are each independently selected from hydrogen, halogen, cyano, C1-C6 alkyl, halo-C1-C6 alkyl, C3-C6 cycloalkyl, C1-C3 alkoxy, alkylsilyl, aryl, substituted aryl, and thioalkyl.

Statement 7: The compound of statement 1 or 6, according to formula (III-1a) or formula (III-2a), or its stereoisomers, pharmaceutically acceptable salts, solvates, deuterated derivatives, metabolites, or prodrugs:

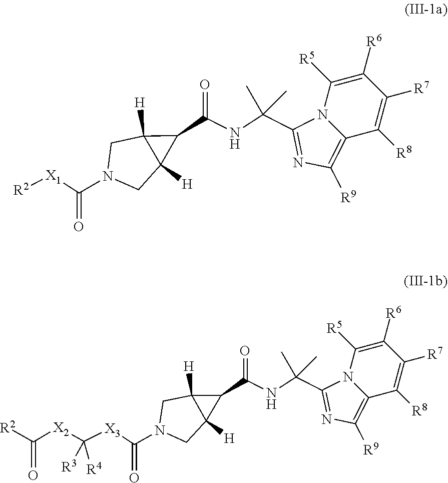

wherein:
   X₁ is selected from a single bond, CH₂, or O;
   X₂ and X₃ are each independently selected from CH₂ or O;
   R² is selected from methyl, ethyl, propyl, isopropyl, butyl, tert-butyl, cyclopropyl, cyclobutyl, cyclopentyl, and cyclohexyl; substituted C1-C6 alkyl or substituted C3-C6 cycloalkyl is further substituted with 1-3 substituents independently selected from the group consisting of fluorine, chlorine, bromine, iodine, cyano, methyl, ethyl, propyl, fluoromethyl, difluoromethyl, trifluoromethyl, cyclopropyl, cyclobutyl, cyclopentyl, methoxy, ethoxy, and propoxy;
   R³ and R⁴ are each independently selected from hydrogen, deuterium, methyl, ethyl, propyl, and isopropyl; and
   R⁵, R⁶, R⁷, R⁸, and R⁹ are each independently selected from hydrogen, fluorine, chlorine, bromine, iodine, methyl, ethyl, propyl, isopropyl, butyl, tert-butyl, cyclopropyl, cyclobutyl, cyclopentyl, cyclohexyl, methylthio, ethylthio, and propylthio.

Statement 8: The compound according to any one of statements 1-7, selected from the following compounds or any one of their stereoisomers, pharmaceutically acceptable salts, solvates, deuterated derivatives, metabolites, or prodrugs:

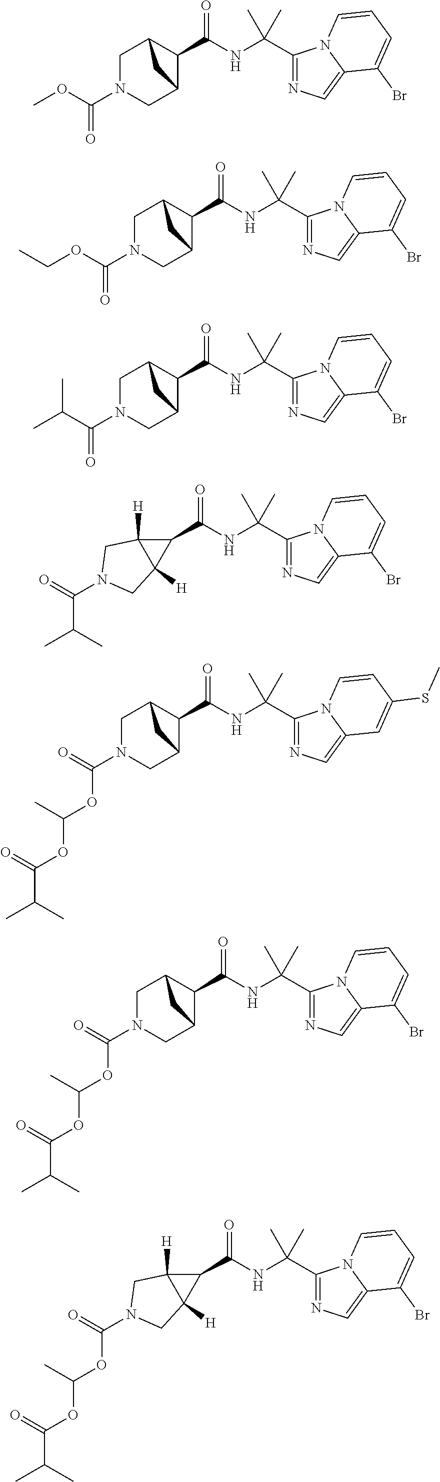

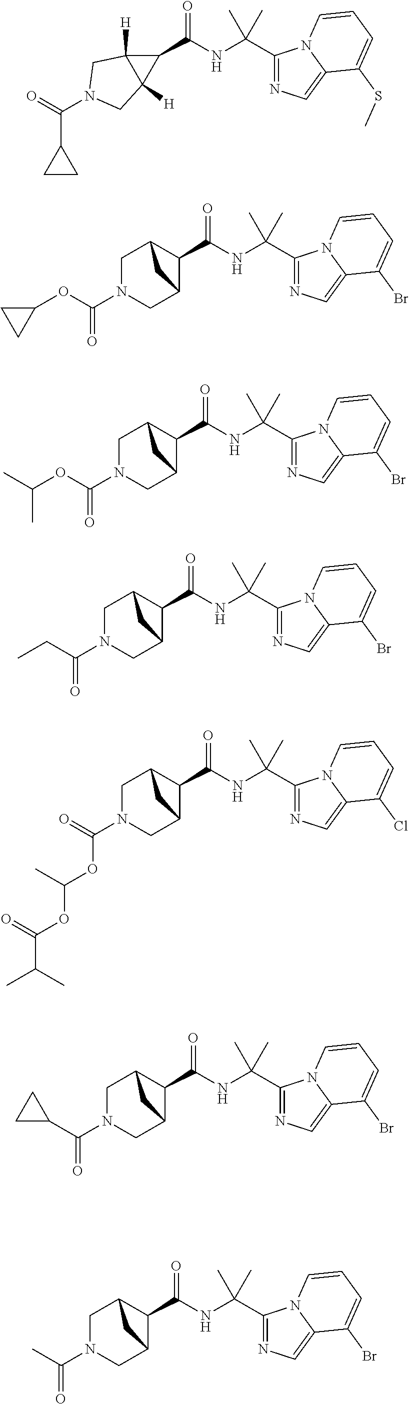

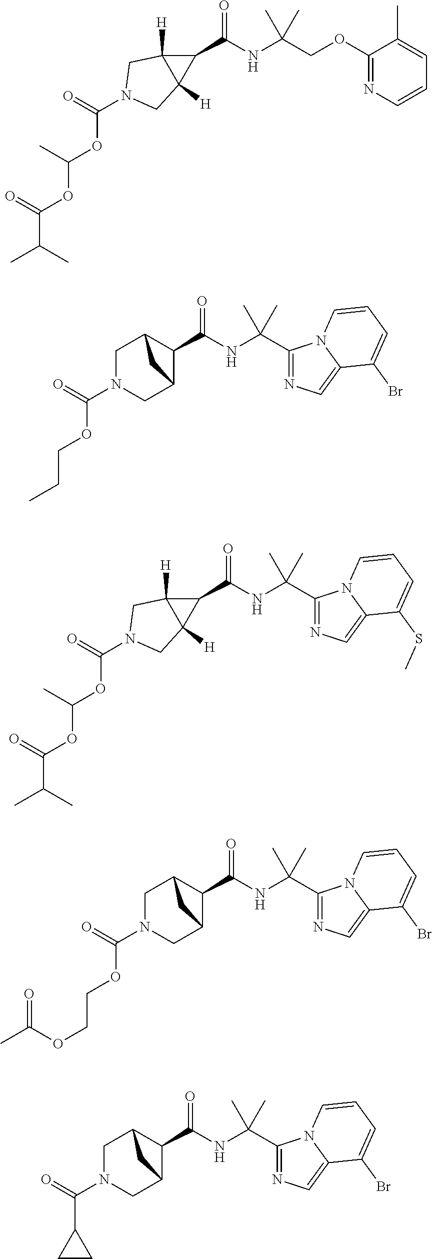 [SMILES: Cc1cccnc1OCC(C)(C)NC(=O)[C@H]1[C@@H]2CN(C(=O)OC(C)OC(=O)C(C)C)C[C@@H]21 ; CCCOC(=O)N1C[C@H]2C[C@@H](C1)[C@@H]2C(=O)NC(C)(C)c1ncc2c(Br)cccn12 ; CSc1cccn2c(C(C)(C)NC(=O)[C@H]3[C@@H]4CN(C(=O)OC(C)OC(=O)C(C)C)C[C@@H]43)ncc12 ; CC(=O)OCCOC(=O)N1C[C@H]2C[C@@H](C1)[C@@H]2C(=O)NC(C)(C)c1ncc2c(Br)cccn12 ; CC(C)(NC(=O)[C@@H]1[C@@H]2C[C@H]1CN(C(=O)C1CC1)C2)c1ncc2c(Br)cccn12]

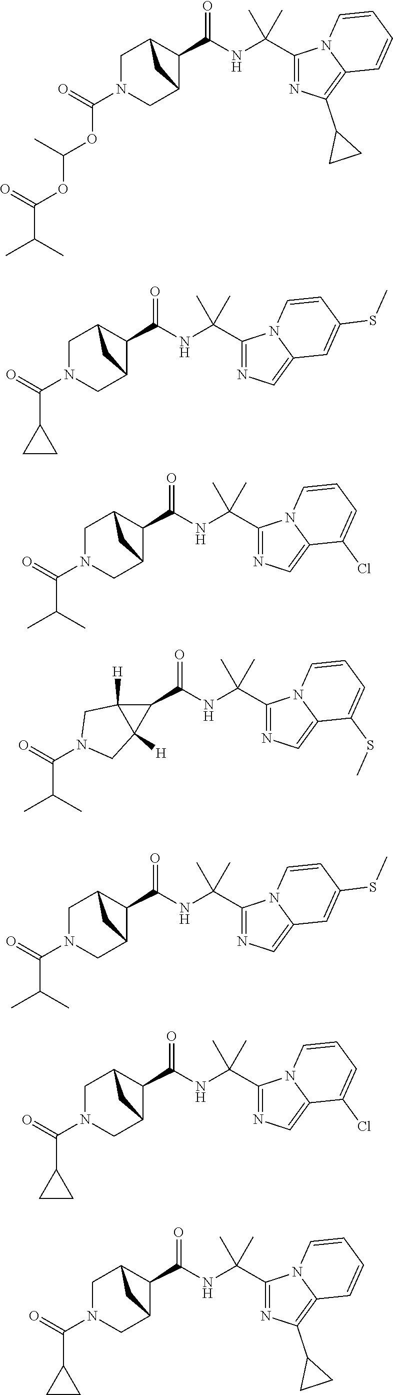

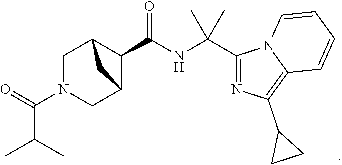 [SMILES: CC(C)C(=O)N1C[C@H]2C[C@@H](C1)[C@@H]2C(=O)NC(C)(C)c1nc(C2CC2)c2ccccn12]

Statement 9: A pharmaceutical composition comprising the compound according to any one of statements 1 to 8, or its stereoisomers, pharmaceutically acceptable salts, solvates, deuterated derivatives, metabolites, or prodrugs, and a pharmaceutically acceptable carrier.

Statement 10: A method of treating or preventing a disease or disorder associated with somatostatin receptor subtype 4 (SSTR4) in a subject in need thereof, the method comprising administering to the subject a therapeutically effective amount of a compound of Formula (I) or a pharmaceutical composition comprising the compound of Formula (I).

Statement 11: A method of treating or preventing a pain condition mediated by somatostatin receptor subtype 4 (SSTR4) in a subject in need thereof, the method comprising administering to the subject a therapeutically effective amount of a compound of Formula (I) or a pharmaceutical composition comprising the compound of Formula (I).

Statement 12: The method of statement 11, wherein the SSTR4-associated pain condition is neuropathic pain or visceral pain.

1. A compound as shown in formula (I), or its stereoisomers, pharmaceutically acceptable salts, solvates, deuterated derivatives, metabolites, or prodrugs: 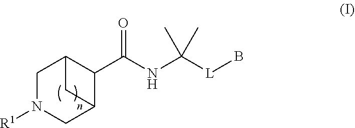
wherein:
n=0 or 1;
L is selected from the group consisting of a bond, —(CH₂)ₘ—, and —(CH₂)ₘO—;
wherein, m=0, 1, 2, or 3;
B is selected from the group consisting of an aryl ring, a substituted aryl ring, a heterocyclic ring, a substituted heterocyclic ring, an alkyl-substituted heterocyclic ring, and a substituted alkyl-substituted heterocyclic ring; the B ring is further optionally substituted with 0-6 substituents independently selected from the group consisting of hydrogen, halogen, cyano, C1-C6 alkyl, halo-C1-C6 alkyl, C3-C6 cycloalkyl, C1-C3 alkoxy, alkylsilyl, aryl, substituted aryl, and thioalkyl;
R¹ is selected from the following: 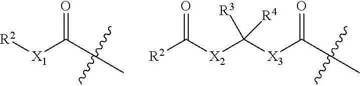
wherein:
X₁ is selected from a single bond, CH₂, or O;
X₂ and X₃ are each independently selected from CH₂ or O;
R² is selected from C1-C10 alkyl, substituted C1-C10 alkyl, C3-C8 cycloalkyl, substituted C3-C8 cycloalkyl, aryl, substituted aryl, heteroaryl, substituted heteroaryl, heterocyclic, substituted heterocyclic; substituted C1-C10 alkyl, substituted C3-C8 cycloalkyl, substituted aryl, heteroaryl, or heterocyclic groups are substituted with 1-3 substituents independently selected from the group consisting of halogen, cyano, C1-C6 alkyl, halo-C1-C6 alkyl, C3-C6 cycloalkyl, and C1-C3 alkoxy; and
R³ and R⁴ are each independently selected from hydrogen, deuterium, and C1-C6 alkyl.

2. The compound of claim 1, according to formula (II) or formula (III), or its stereoisomers, pharmaceutically acceptable salts, solvates, deuterated derivatives, metabolites, or prodrugs: 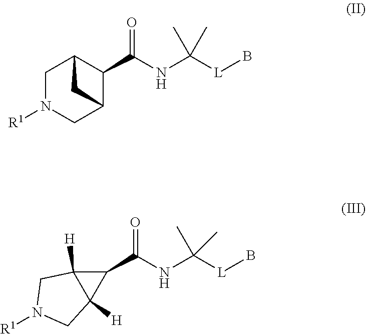
wherein:
L is selected from the group consisting of —(CH₂)ₘ— and —(CH₂)ₘO—;
wherein m is 0 or 1;
B is selected from the group consisting of an aryl ring, substituted aryl ring, heterocyclic ring, substituted heterocyclic ring, alkyl-substituted heterocyclic ring, and substituted alkyl-substituted heterocyclic ring; the B ring is further optionally substituted with 0-6 substituents independently selected from the group consisting of hydrogen, halogen, cyano, C1-C6 alkyl, halo-C1-C6 alkyl, C3-C6 cycloalkyl, C1-C3 alkoxy, alkylsilyl, aryl, substituted aryl, and thioalkyl;
R¹ is selected from the following: 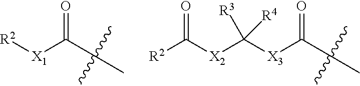
wherein:
X₁ is selected from a single bond, CH₂, or O;
X₂ and X₃ are each independently selected from CH₂ or O;
R² is selected from C1-C10 alkyl, C3-C8 cycloalkyl, substituted C1-C10 alkyl, or substituted C3-C8 cycloalkyl; substituted C1-C10 alkyl or substituted C3-C8 cycloalkyl is further substituted with 1-3 substituents independently selected from the group consisting of halogen, cyano, C1-C6 alkyl, halo-C1-C6 alkyl, C3-C6 cycloalkyl, and C1-C3 alkoxy; and
R³ and R⁴ are each independently selected from hydrogen, deuterium, and C1-C3 alkyl.

3. The compound of claim 2, wherein B is selected from phenyl, naphthyl, pyridyl, pyrimidyl, and imidazopyridyl.

4. The compound of claim 1, according to formula (II-1), or its stereoisomers, pharmaceutically acceptable salts, solvates, deuterated derivatives, metabolites, or prodrugs: 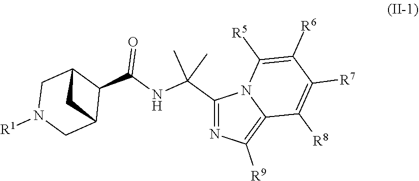
R¹ is selected from the following: 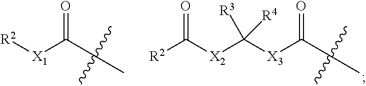
wherein:
X₁ is selected from a single bond, CH₂, or O;
X₂ and X₃ are each independently selected from CH₂ or O;
R² is selected from C1-C6 alkyl, C3-C6 cycloalkyl, substituted C1-C6 alkyl, or substituted C3-C6 cycloalkyl; substituted C1-C6 alkyl or substituted C3-C6 cycloalkyl is further substituted with 1-3 substituents independently selected from the group consisting of halogen, cyano, C1-C6 alkyl, halo-C1-C6 alkyl, C3-C6 cycloalkyl, and C1-C3 alkoxy;
R³ and R⁴ are each independently selected from hydrogen, deuterium, and C1-C3 alkyl; and
R⁵, R⁶, R⁷, R⁸, and R⁹ are each independently selected from hydrogen, halogen, cyano, C1-C6 alkyl, halo-C1-C6 alkyl, C3-C6 cycloalkyl, C1-C3 alkoxy, alkylsilyl, aryl, substituted aryl, and thioalkyl.

5. The compound of claim 1, according to formula (II-1a) or formula (II-1b), or its stereoisomers, pharmaceutically acceptable salts, solvates, deuterated derivatives, metabolites, or prodrugs: 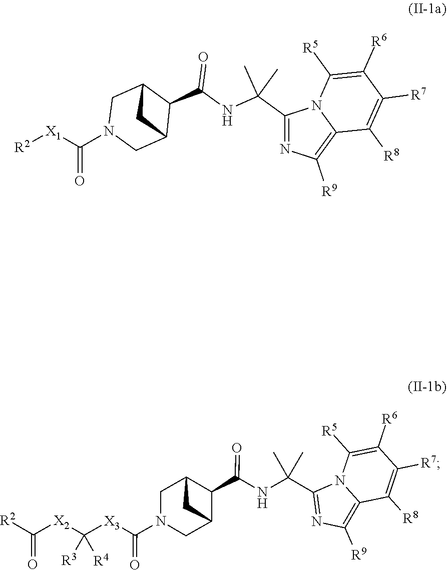
R¹ is selected from the following: 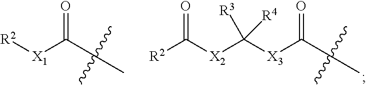
wherein,
X₁ is selected from a single bond, CH₂, or O;
X₂ and X₃ are each independently selected from CH₂ or O;
R² is selected from methyl, ethyl, propyl, isopropyl, butyl, tert-butyl, cyclopropyl, cyclobutyl, cyclopentyl, cyclohexyl; substituted C1-C6 alkyl or substituted C3-C6 cycloalkyl is further substituted with 1-3 substituents independently selected from the group consisting of fluorine, chlorine, bromine, iodine, cyano, methyl, ethyl, propyl, fluoromethyl, difluoromethyl, trifluoromethyl, cyclopropyl, cyclobutyl, cyclopentyl, methoxy, ethoxy, and propoxy;
R³ and R⁴ are each independently selected from hydrogen, deuterium, methyl, ethyl, propyl, and isopropyl;
R⁵, R⁶, R⁷, R⁸, and R⁹ are each independently selected from hydrogen, fluorine, chlorine, bromine, iodine, methyl, ethyl, propyl, isopropyl, butyl, tert-butyl, cyclopropyl, cyclobutyl, cyclopentyl, cyclohexyl, methylthio, ethylthio, and propylthio.

6. The compound of claim 1, according to formula (III-1) or formula (III-2), or its stereoisomers, pharmaceutically acceptable salts, solvates, deuterated derivatives, metabolites, or prodrugs: 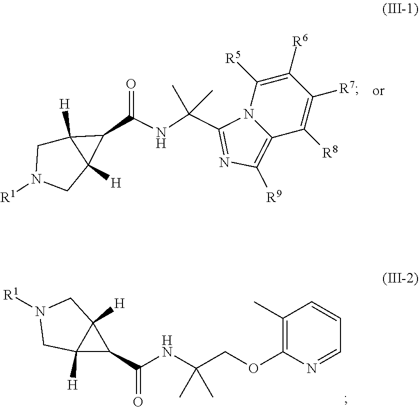
R¹ is selected from the following: 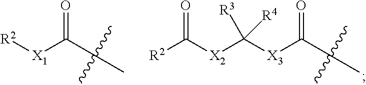
wherein:
X₁ is selected from a single bond, CH₂, or O;
X₂ and X₃ are each independently selected from CH₂ or O;
R² is selected from C1-C6 alkyl, C3-C6 cycloalkyl, substituted C1-C6 alkyl, or substituted C3-C6 cycloalkyl; substituted C1-C6 alkyl or substituted C3-C6 cycloalkyl is further substituted with 1-3 substituents independently selected from the group consisting of halogen, cyano, C1-C6 alkyl, halo-C1-C6 alkyl, C3-C6 cycloalkyl, and C1-C3 alkoxy;
R³ and R⁴ are each independently selected from hydrogen, deuterium, and C1-C3 alkyl; and
R⁵, R⁶, R⁷, R⁸, and R⁹ are each independently selected from hydrogen, halogen, cyano, C1-C6 alkyl, halo-C1-C6 alkyl, C3-C6 cycloalkyl, C1-C3 alkoxy, alkylsilyl, aryl, substituted aryl, and thioalkyl.

7. The compound of claim 1, according to formula (III-1a) or formula (III-2a), or its stereoisomers, pharmaceutically acceptable salts, solvates, deuterated derivatives, metabolites, or prodrugs: 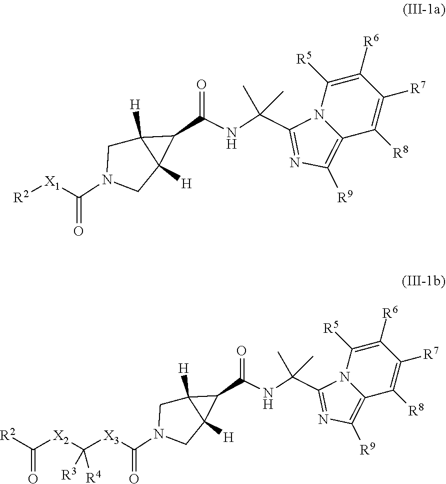
wherein:
X₁ is selected from a single bond, CH₂, or O;
X₂ and X₃ are each independently selected from CH₂ or O;
R² is selected from methyl, ethyl, propyl, isopropyl, butyl, tert-butyl, cyclopropyl, cyclobutyl, cyclopentyl, and cyclohexyl; substituted C1-C6 alkyl or substituted C3-C6 cycloalkyl is further substituted with 1-3 substituents independently selected from the group consisting of fluorine, chlorine, bromine, iodine, cyano, methyl, ethyl, propyl, fluoromethyl, difluoromethyl, trifluoromethyl, cyclopropyl, cyclobutyl, cyclopentyl, methoxy, ethoxy, and propoxy;
R³ and R⁴ are each independently selected from hydrogen, deuterium, methyl, ethyl, propyl, and isopropyl; and
R⁵, R⁶, R⁷, R⁸, and R⁹ are each independently selected from hydrogen, fluorine, chlorine, bromine, iodine, methyl, ethyl, propyl, isopropyl, butyl, tert-butyl, cyclopropyl, cyclobutyl, cyclopentyl, cyclohexyl, methylthio, ethylthio, and propylthio.

8. The compound of claim 1, wherein the compound is selected from the following compounds or any one of their stereoisomers, pharmaceutically acceptable salts, solvates, deuterated derivatives, metabolites, or prodrugs:

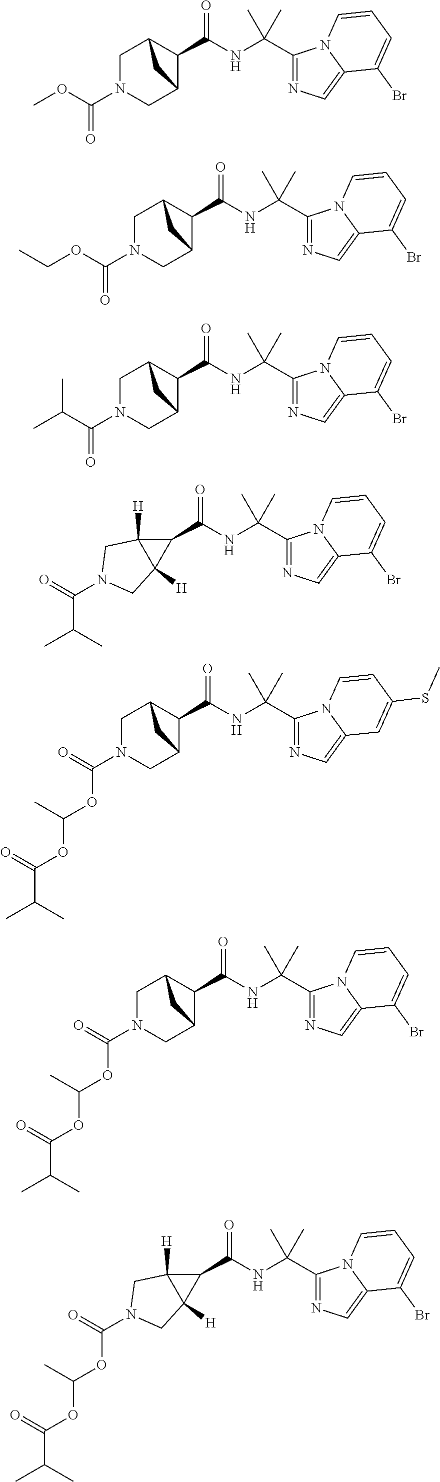

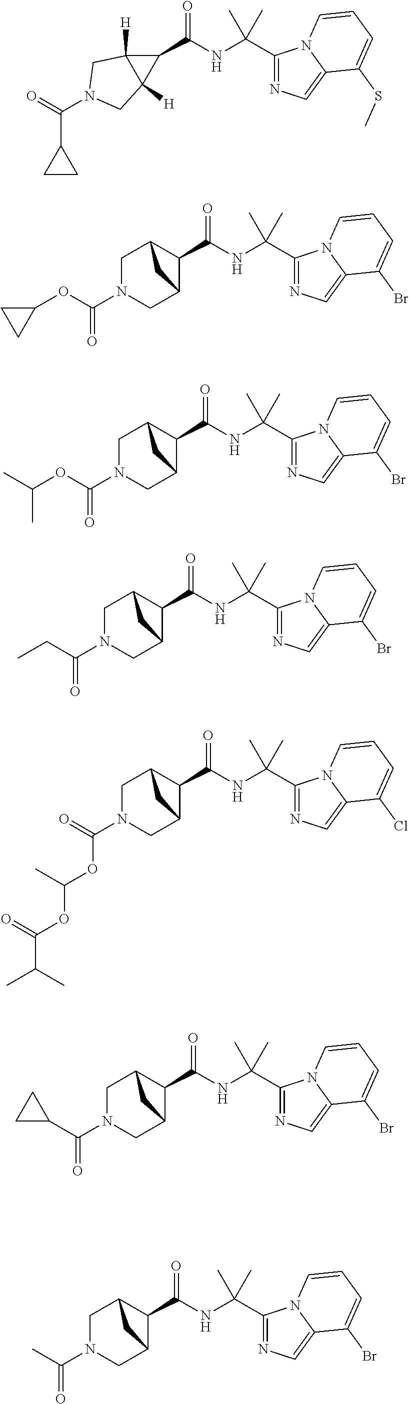

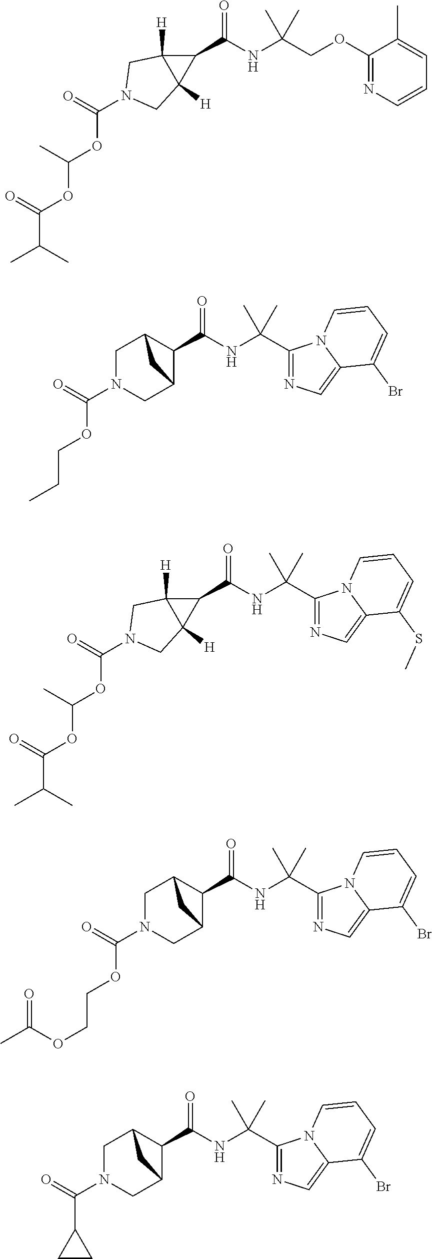 [SMILES: Cc1cccnc1OCC(C)(C)NC(=O)[C@H]1[C@@H]2CN(C(=O)OC(C)OC(=O)C(C)C)C[C@@H]21 ; CCCOC(=O)N1C[C@H]2C[C@@H](C1)[C@@H]2C(=O)NC(C)(C)c1ncc2c(Br)cccn12 ; CSc1cccn2c(C(C)(C)NC(=O)[C@H]3[C@@H]4CN(C(=O)OC(C)OC(=O)C(C)C)C[C@@H]43)ncc12 ; CC(=O)OCCOC(=O)N1C[C@H]2C[C@@H](C1)[C@@H]2C(=O)NC(C)(C)c1ncc2c(Br)cccn12 ; CC(C)(NC(=O)[C@@H]1[C@@H]2C[C@H]1CN(C(=O)C1CC1)C2)c1ncc2c(Br)cccn12]

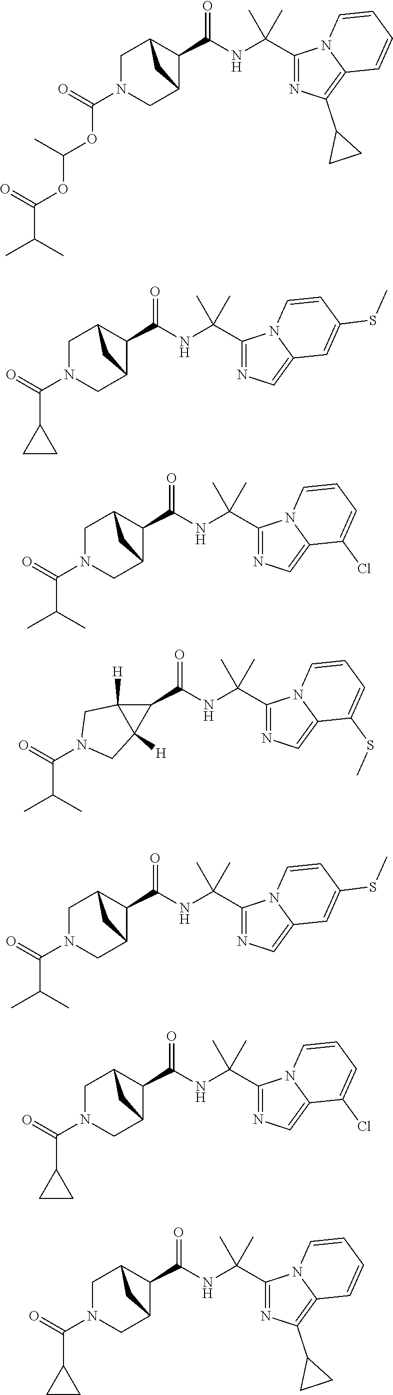

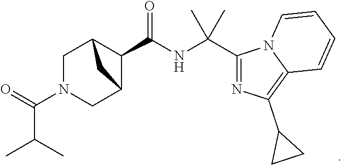 [SMILES: CC(C)C(=O)N1C[C@H]2C[C@@H](C1)[C@@H]2C(=O)NC(C)(C)c1nc(C2CC2)c2ccccn12]

9. A pharmaceutical composition comprising the compound according to claim 1, or its stereoisomers, pharmaceutically acceptable salts, solvates, deuterated derivatives, metabolites, or prodrugs, and a pharmaceutically acceptable carrier.

10. A method of treating or preventing a disease or disorder associated with somatostatin receptor subtype 4 (SSTR4) in a subject in need thereof, the method comprising administering to the subject a therapeutically effective amount of a compound of Formula (I) or a pharmaceutical composition comprising the compound of Formula (I).

11. A method of treating or preventing a pain condition mediated by somatostatin receptor subtype 4 (SSTR4) in a subject in need thereof, the method comprising administering to the subject a therapeutically effective amount of a compound of Formula (I) or a pharmaceutical composition comprising the compound of Formula (I).

12. The method of claim 11, wherein the SSTR4-associated pain condition is neuropathic pain or visceral pain.
